SEQUENCE LISTING (2 sequences)

SEQ ID NO: 1 - protein, 581 aa, Homo sapiens
MAVRQALGRGLQLGRALLLRFTGKPGRAYGLGRPGPAAGCVRGERPGWAAGPGAEPRRVGLGLPNRLRFFRQSVAGLAARLQRQFVVRAWGCAGPCGRAVFLAFGLGLGLIEEKQAESRRAVSACQEIQAIFTQKSKPGPDPLDTRRLQGFRLEEYLIGQSIGKGCSAAVYEATMPTLPQNLEVTKSTGLLPGRGPGTSAPGEGQERAPGAPAFPLAIKMMWNISAGSSSEAILNTMSQELVPASRVALAGEYGAVTYRKSKRGPKQLAPHPNIIRVLRAFTSSVPLLPGALVDYPDVLPSRLHPEGLGHGRTLFLVMKNYPCTLRQYLCVNTPSPRLAAMMLLQLLEGVDHLVQQGIAHRDLKSDNILVELDPDGCPWLVIADFGCCLADESIGLQLPFSSWYVDRGGNGCLMAPEVSTARPGPRAVIDYSKADAWAVGAIAYEIFGLVNPFYGQGKAHLESRSYQEAQLPALPESVPPDVRQLVRALLQREASKRPSARVAANVLHLSLWGEHILALKNLKLDKMVGWLLQQSAATLLANRLTEKCCVETKMKMLFLANLECETLCQAALLLCSWRAAL

SEQ ID NO: 2 - protein, 570 aa, Homo sapiens
MSVRAVGSRLFKHGRSLIQQFCKRDLNTTIGDKINAVSQATAAPSSLPKTQIPKNFALRNVGVQLGLQARRILIDNVLNRVTNSLSAELRKKATRRILFGDSAPFFALVGVSIASGTGILTKEEELEGVCWEIREAISKIKWQYYDIDESRFESNPITLNDLSLGKPIAKGTNGVVYSAKVKDDETDDNKYPFALKMMFNYDIQSNSMEILKAMYRETVPARMYYSNHDLNNWEIELANRRKHLPPHPNIVAIFSVFTDLIQELEGSKDLYPAALPPRLHPEGEGRNMSLFLLMKRYDCNLQSFLSTAPSTRTSLLLLAQLLEGVAHMTAHGIAHRDLKSDNLLLDTSEPESPILVISDFGCCLADKTNGLSLPYTSYEMDKGGNTALMAPEIICQKPGTFSVLNYSKADLWAVGAIAYEIFNCHNPFYGPSRLKNFNYKEGDLPKLPDEVPTVIQALVANLLKRNPNKRLDPEVAANVCQLFLWAPSTWLKPGLKVPTSGEILQWLLSLTTKVLCEGKINNKSFGEKFTRNWRRTYPEYLLISSFLCRAKLANVRNALHWIQENLPELD

(19) United States
(12) Patent Application Publication
(10) Pub. No.: US 2026/0014184 A1
(43) Pub. Date: Jan. 15, 2026

(54) ADENOSINE DERIVATIVES FOR USE IN THE TREATMENT OF NEURODEGENERATIVE DISORDERS AND CANCER

(71) Applicant: UNIVERSITY COLLEGE CARDIFF CONSULTANTS LIMITED, Cardiff, South Wales (GB)

(72) Inventor: Youcef MEHELLOU, Cardiff (GB)

(21) Appl. No.: 19/139,661

(22) PCT Filed: Dec. 12, 2023
(86) PCT No.: PCT/GB2023/053191

§ 371 Date: Jun. 16, 2025

(30) Foreign Application Priority Data
Dec. 15, 2022 (GB) 2218917.9

(51) Int. Cl. A61K 31/7076; A61P 25/16; A61P 25/28; C07H 1/00; C07H 19/167

(52) U.S. Cl. CPC A61K 31/7076; A61P 25/16; A61P 25/28; C07H 1/00; C07H 19/167

(57) ABSTRACT

The invention relates to a compound of General Formula (I): (General Formula I) wherein R¹ and R² are as defined herein: for use in the treatment of neurodegenerative diseases or conditions that are characterised by an elevated level of Ubiquitin Ser65 phosphorylation and/or in the treatment of cancer. The invention also relates to pharmaceutical compositions and combination therapeutic agents comprising compounds of formula (I) for treating these conditions as well to a method of treating such diseases and conditions using the compounds of General Formula (I).

### FIELD OF THE INVENTION

The invention relates to a therapeutic agent for use in the treatment of neurodegenerative diseases or conditions that are characterised by an elevated level of Ubiquitin Ser65 phosphorylation and/or cancer, and a method of treating such diseases or conditions wherein the therapeutic agent or composition or combination therapeutic according to the invention is administered to a subject having, or suspected of having a neurodegenerative disease or condition that is characterised by an elevated level of Ubiquitin Ser65 phosphorylation and/or cancer.

### BACKGROUND OF THE INVENTION

Parkinson's disease belongs to a group of conditions called motor system disorders, which are the result of the loss of dopamine-producing brain cells. Parkinson's disease (PD) is the second leading cause of neurodegeneration in man and to date there are no treatments that can slow or halt the clinical progression.

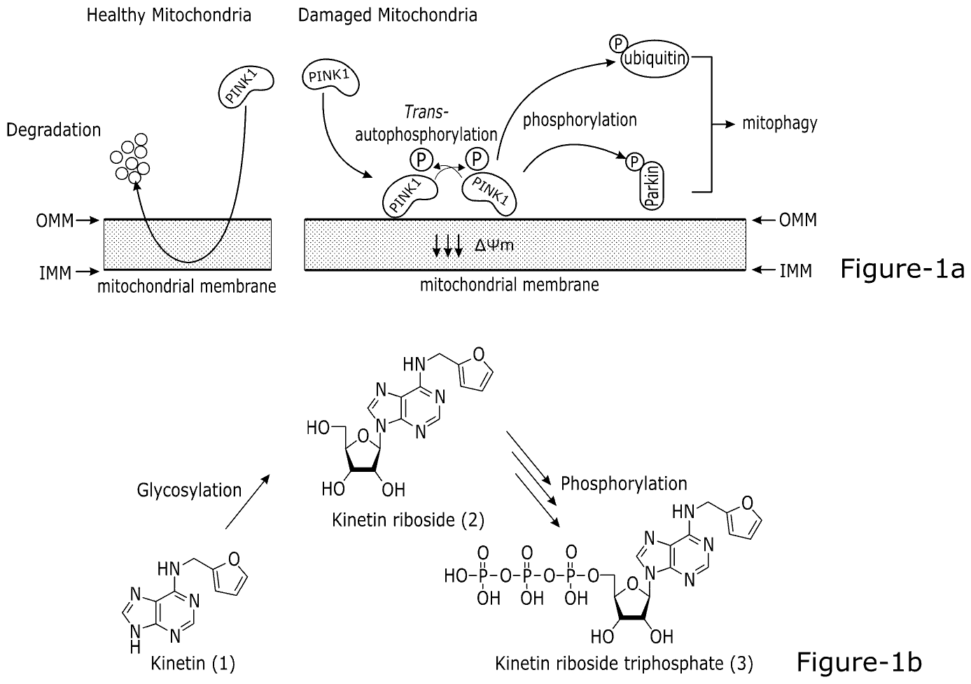

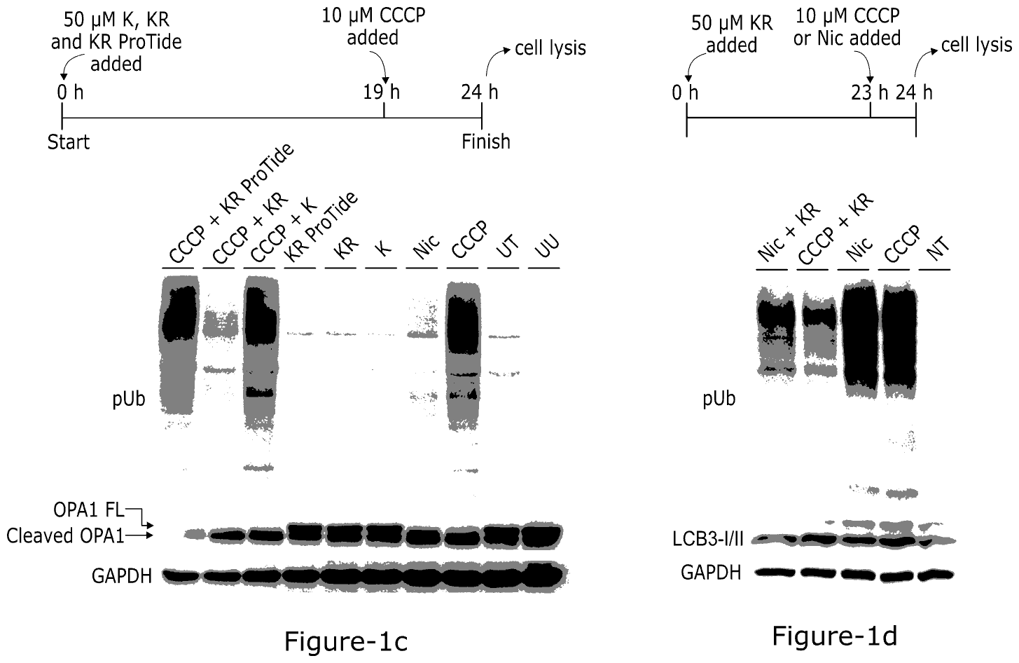

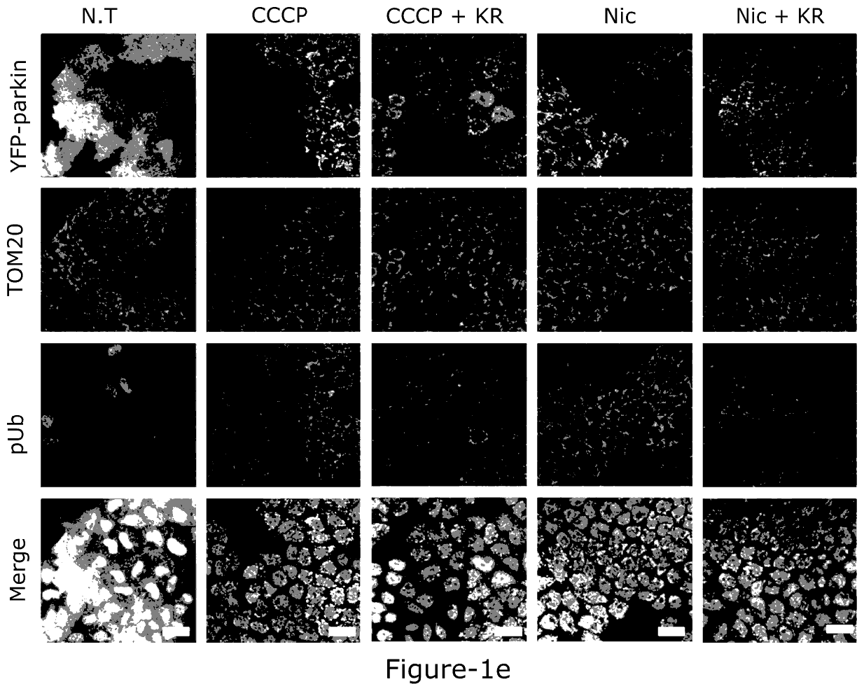

The mitochondria play an important role in the pathogenesis of PD, and also other neurodegenerative diseases and cardiomyopathy, and in relation to this the mitochondrial serine/threonine PTEN-induced kinase 1 (PINK1) has emerged as a key player in mitochondrial quality control. In healthy mitochondria, PINK1 is constitutively recruited to the mitochondrial membrane where it undergoes N-terminal cleavage by proteases and subsequent proteasomal degradation in the cytosol (FIG. 1a). However, in damaged mitochondria and following depolarization of the inner mitochondrial membrane, PINK1 gets stabilized on the outer mitochondrial membrane (OMM) in its full-length form. Accumulation of PINK1 results in trans-autophosphorylation and its subsequent activation. Active PINK1 then phosphorylates the E3 ubiquitin ligase parkin at serine 65 and ubiquitin also at serine 65 (Ub pSer65). This ultimately results in the ubiquitylation of various proteins on the OMM, resulting in mitochondrial degradation by the autophagic machinery, a process termed mitophagy.

As such, activating PINK1 to trigger mitophagy, has been commonly proposed as a potential new therapeutic target for Parkinson's disease. Specifically, PINK1 kinase activity has been highlighted as being vital in preventing the development of neurodegeneration, exemplified by its loss-of-function mutations leading to a form of early-onset Parkinson's disease (PD). This observation led to the discovery that kinetin, an N⁶-substituted adenine (1, FIG. 1b), enhanced PINK1 activation in cells when exposed to the de-polarizing agent carbonyl cyanide m-chlorophenyl-hydrazine (CCCP), an agent used to stimulate mitochondrial damage. Kinetin activation of PINK1 was noted to be due to its bioconversion to the active metabolite kinetin riboside triphosphate (3, FIG. 1b), which acts as an ATP-neosubstrate for PINK1. With this observation in mind, and as a result of our interest in developing nucleoside analogue therapeutics, we subsequently showed that the nucleoside derivative of kinetin, called kinetin riboside 2, exhibited more potent CCCP-independent activation of PINK1 in cells compared to its nucleobase derivative, kinetin, as evidenced by parkin Ser65 phosphorylation.

Therefore, the phosphorylation of ubiquitin by the mitochondrial protein kinase PINK1, upon mitochondrial depolarization, is typically regarded an important step in the repair and recycling of the mitochondria via autophagy, and so activators of PINK1 deemed to be promising therapeutics in treatment of many forms of PD.

However, recent evidence in post-mortem analysis of brain tissues of PD and dementia sufferers with no discernible PINK1 genetic causative factors, such as Lewy Body dementia and idiopathic forms of PD (i.e. aged and sporadic PD patients), have shown the converse to be true with elevated levels of Ub Ser65 phosphorylation^{9,10}. Accordingly, these subsets of PD patients, and dementia sufferers, exhibit increased levels of phospho-ubiqutin (Ubiquitin Ser65 phosphorylation) in the brain. This therefore means that conventional activators of PINK1, and so associated increased phosphorylation of Ubiquitin, have no effect in such patients where elevated Ub Ser65 phosphorylation is apparent. Further, and whilst the role of mitophagy in tumorigenesis has not yet been fully elucidated, a significant body of evidence has shown that dysregulation of mitophagy is frequently associated with many types of cancer, with the accumulation of dysfunctional mitochondria contributing to tumorigenesis.

We herein disclose that in cells and astrocytes, N⁶ substituted adenosines, such as N⁶-(2-furanylmethyl) adenosine (known as kinetin riboside) and N⁶-benzyladenosine, unexpectedly inhibit ubiquitin phosphorylation that is induced by established mitochondrial depolarizing agents, CCCP and niclosamide. Although these nucleoside analogues inhibited niclosamide- and CCCP-induced ubiquitin phosphorylation, they did not prevent the mitochondrial membrane depolarization. Notably, the treatment of cells with these nucleoside analogues alone induced low level mitophagy and did not cause mitochondrial fragmentation. Together, this work presents N⁶-substituted adenosines as new inhibitors of PINK1-mediated ubiquitin phosphorylation and highlights their potential utility in unexpectedly treating aged and sporadic PD, as well as Lewy body dementia patients, who have elevated levels of phosphorylated ubiquitin and wherein conventional activators of PINK1 activity would have no effect, and/or in the treatment of cancer. Our findings suggest that these compounds and their analogues hold promise as therapeutics to halt the progression of neuronal death in idiopathic Parkinson's Disease and related disorders, and/or halt the progression of tumorigenesis resulting from an accumulation of dysfunctional mitochondria.

### STATEMENTS OF INVENTION

The present invention, in its various aspects, is as set out in the accompanying claims.

According to a first aspect, the invention provides a compound of General Formula (I), including all tautomers thereof:

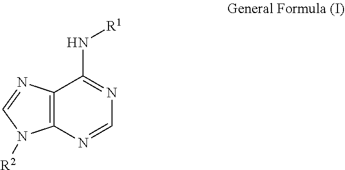

wherein:
   R¹ is a C₁₋₁₀ alkyl, C₃₋₁₀ cycloalkyl, C₆₋₁₀ aryl, heterocycloalkyl or heteroaryl group, optionally substituted with one or more substituents selected from OH, halo, nitro, C₁₋₄ alkyl, C₁₋₄ haloalkyl, —O(C₁₋₄ alkyl), —O(C₁₋₄ haloalkyl), NH₂, NH(C₁₋₄ alkyl) and N(C₁₋₄ alkyl)₂; and
   R² is a furanose moiety of General Formula (II):

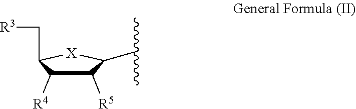

wherein:
X is O, NH, S or CH₂;
R³ is H, OH, halo, nitro, C₁₋₄ alkyl, C₁₋₄ haloalkyl, —O(C₁₋₄ alkyl), —O(C₁₋₄ haloalkyl), NH₂, NH(C₁₋₄ alkyl) and N(C₁₋₄ alkyl)₂, or a mono-, di- or tri-phosphate derivate of General Formula (VIII), wherein q is 0, 1 or 2, and each R¹¹ is independently selected from OH or an aryloxy, amino acid ester or pivaloyloxymethyl masking group;

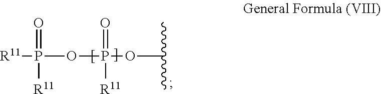

and
R⁴ and R⁵ is independently selected from H, OH, halo, nitro, C₁₋₄ alkyl, C₁₋₄ haloalkyl, —O(C₁₋₄ alkyl), —O(C₁₋₄ haloalkyl), NH₂, NH(C₁₋₄ alkyl) and N(C₁₋₄ alkyl)₂,
or a pharmaceutically or veterinarily acceptable salt or hydrate thereof,
for use in the treatment of a neurodegenerative disease or condition that is characterised by an elevated level of Ubiquitin Ser65 phosphorylation and/or cancer.

Compounds of general formula (1) have been shown to inhibit PINK1-mediated ubiquitin phosphorylation, and so are useful for treating disorders and conditions that are associated with elevated levels of phosphorylated ubiquitin and so conventional activators of PINK1 activity would have no effect.

The compounds of General Formula (I) are particularly useful for the treatment of idiopathic (i.e. aged and/or sporadic) Parkinson's disease, the treatment of Lewy body dementia and/or cancer. More particularly, the compounds of General Formula (I) are useful for the treatment of idiopathic Parkinson's disease and/or Lewy body dementia.

In the present invention the term “C₁₋₁₀ alkyl” refers to refers to a straight or branched saturated hydrocarbon group having one to ten carbon atoms. Examples include methyl, ethyl, n-propyl, isopropyl, t-butyl, and n-hexyl. As is readily appreciated, other alkyl groups are as defined above but have different numbers of carbon atoms. For example, “C₁₋₄ alkyl” has from 1 to 4 carbon atoms.

The term “C₃₋₁₀ cycloalkyl” refers, in the context of the specification, to a cyclic, saturated hydrocarbon group comprising from 3 to 10 carbon atoms and containing a single ring or multiple fused rings.

The term “C₆₋₁₀ aryl” refers to a ring system with aromatic character having from 6 to 10 ring carbon atoms and containing a single ring or multiple fused rings. Where an aryl group contains two fused rings, both rings need not be fully aromatic in character. Examples of aromatic moieties are phenyl and naphthyl.

The term “heterocycloalkyl” in the context of the specification refers to a saturated ring system having from 3 to 10 ring atoms (unless otherwise specified), at least one, and optionally two or three, of which is a heteroatom selected from N, O and S, and containing a single ring or multiple fused rings.

The term “heteroaryl” in the context of the specification refers to a ring system with aromatic character having from 6 to 10 ring atoms (unless otherwise specified), at least one, and optionally two or three, of which is a heteroatom selected from N, O and S, and containing a single ring or multiple fused rings. Where a heteroaryl group contains more than one ring, not all rings must be fully aromatic in character.

Examples of heteroaryl groups include pyridine, pyrimidine, indole, pyrrole, imidazole, triazole, tetrazole, oxazole, thiazole, benzofuran, benzimidazole and indoline.

In the present specification “halo” refers to fluoro, chloro, bromo or iodo, and more suitably to chloro or bromo.

In the present specification “C₁₋₄ haloalkyl” refers to a C₁₋₄ alkyl group substituted with one or more halo atoms, up to per-substitution. Examples include chloromethyl, trifluoromethyl, 2-chloroethyl, 1-bromoethyl etc.

The term “aryloxy” refers, in the context of the specification, to a masking group of General Formula (IX-A), wherein R¹² is a C₅₋₂₅ aryl or a 5 to 25 membered heteroaryl group, either of which is optionally substituted with one or more functional groups selected from hydroxy, thiol, thioether, alkoxy and amino:

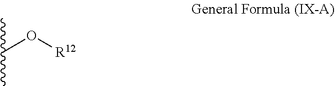

The term “amino acid ester” refers, in the context of the specification, to a masking group of General Formula (IX-B), wherein: R¹³ is H, or a saturated or unsaturated hydrocarbon, preferably a C₁₋₄ alkyl, chain which is optionally substituted with one or more functional groups selected from thiol, thioether, alkoxy and amino; and R¹⁴ is a saturated or unsaturated hydrocarbon, preferably a C₁₋₄ alkyl chain or C₆ aryl group, which is optionally substituted with one or more functional groups selected from hydroxy, thiol, thioether, alkoxy and amino:

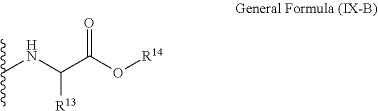

The term “pivaloyloxymethyl” refers, in the context of the specification, to a masking group of General Formula (IX-C), wherein each of R¹⁵ and R¹⁶ is independently hydrogen, halo or a C₁₋₁₀ alkyl group, optionally substituted with one or more substituents selected from hydroxy, thiol, thioether, alkoxy and amino, or R¹⁵ and R¹⁶, together with the carbon to which R¹⁵ and R¹⁶ are attached, form a C₃₋₄ cycloalkyl group or 3 or 4 membered heterocycloalkyl group:

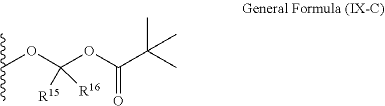

Salts of the compounds of general formula (I) are suitably pharmaceutically or veterinarily acceptable salts. Depending on the nature of R¹ to R⁵, these may be basic addition salts such as sodium, potassium, calcium, aluminium, zinc, magnesium and other metal salts as well as choline, diethanolamine, ethanolamine, ethyl diamine, megulmine and other well known basic addition salts as summarised in Paulekuhn et al., (2007) J. Med. Chem. 50: 6665-6672 and/or known to those skilled in the art. Alternatively, when the compound of General Formula (I) contains an amino group, this may be quaternised to form a salt with a counter ion such as halide, hydroxide, sulfate, nitrate, phosphate, formate, acetate, trifluoroacetate, fumarate, citrate, tartrate, oxalate, succinate, mandelate, methane sulfonate and p-toluene sulfonate.

In compounds of general formula (1), R¹ is preferably substituted a C₁₋₆ alkyl, C₃₋₆ cycloalkyl or C₆ aryl, optionally substituted with one or more substituents selected from OH and C₁₋₄ alkyl.

More preferably R¹ is a group of General Formula (III-A), (III-B), (III-C), (III-D), (III-E) or (III-F):

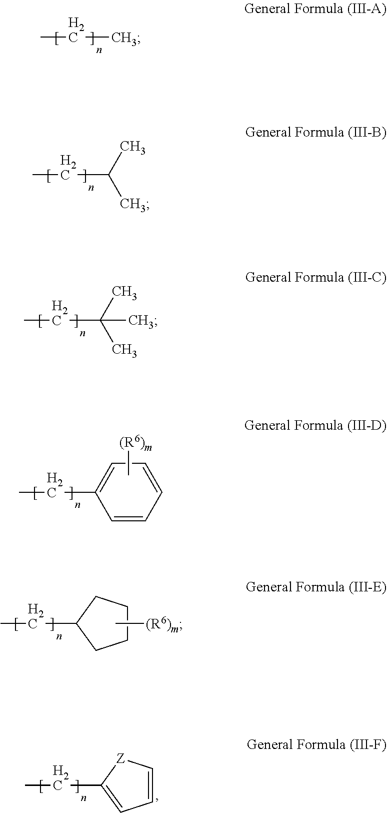

wherein:
   n is 0,1, 2 or 3;
   m is 0, 1, 2 or 3;
   Z is O, NH, S or CH₂, and is preferably CH₂; and
   R⁶ is OH or O(C₁₋₄alkyl).

Most preferably R¹ is selected from:

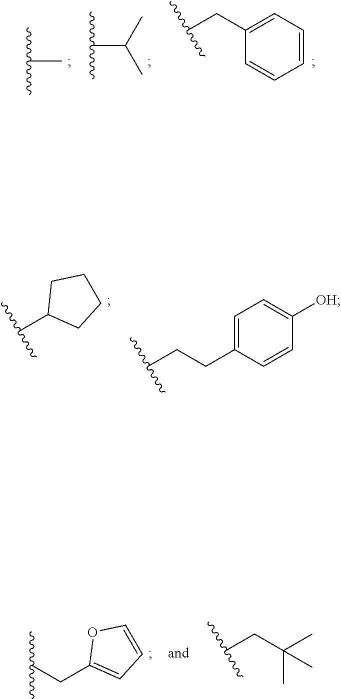

As noted above, the compounds of General Formula (I) comprise a furanose moiety of General Formula (II). In preferred embodiments, X is O and/or R⁴ and R⁵ are independently selected from H and OH, and/or R³ is H, OH or a mono-, di- or tri-phosphate derivative of General Formula (VIII).

In some compounds of the invention, each of R³, R⁴ and R⁵ are OH. As would be readily appreciated by the skilled reader, such compounds are adenosine ribonucleoside analogues.

In other suitable compounds both R³ and R⁴ are hydroxyl, and R⁵ is H. As would be readily appreciated by the skilled reader, such compounds are adenosine deoxyribonucleoside analogues.

Exemplary compounds of General Formula (I) include the known compound kinetin riboside, which has the structure of Formula (IV):

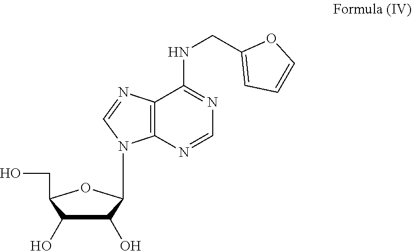

Additional exemplary compounds of General Formula (I) include the following novel N⁶-substituted adenosine analogues:

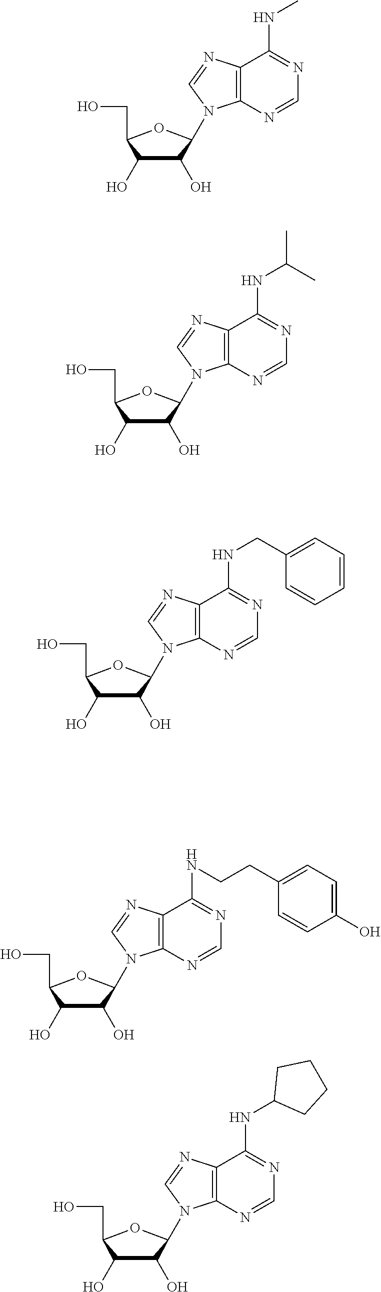

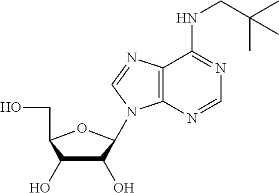

In particularly preferred embodiments, the compound of General Formula (I) is selected from the following structures:

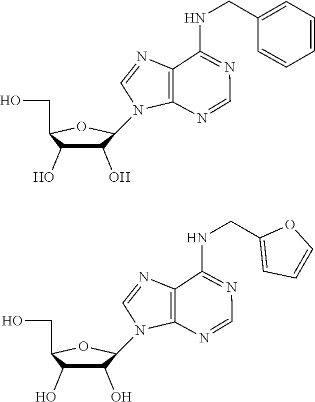

As noted above, the known compound kinetin riboside is a compound of General Formula (I) as defined above. Therefore, according to a second aspect, the invention provides a compound of General Formula (I), including all tautomers thereof:

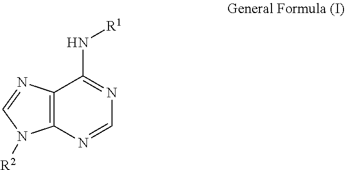

wherein:
   R¹ is a C₁₋₁₀ alkyl, C₃₋₁₀ cycloalkyl, C₆₋₁₀ aryl, heterocycloalkyl or heteroaryl group, optionally substituted with one or more substituents selected from OH, halo, nitro, C₁₋₄ alkyl, C₁₋₄ haloalkyl, —O(C₁₋₄ alkyl), —O(C₁₋₄ haloalkyl), NH₂, NH(C₁₋₄ alkyl) and N(C₁₋₄ alkyl)₂; and
   R² is a furanose moiety of General Formula (II):

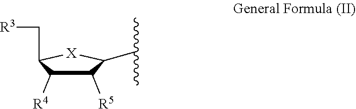

wherein:
   X is O, NH, S or CH₂;
   R³ is H, OH, halo, nitro, C₁₋₄ alkyl, C₁₋₄ haloalkyl, —O(C₁₋₄ alkyl), —O(C₁₋₄ haloalkyl), NH₂, NH(C₁₋₄ alkyl) and N(C₁₋₄ alkyl)₂, or a mono-, di- or tri-phosphate derivate of General Formula (VIII), wherein q is 0, 1 or 2, and each R¹¹ is independently selected from OH or an aryloxy, amino acid ester or pivaloyloxymethyl masking group;

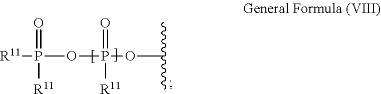

and
R⁴ and R⁵ is independently selected from H, OH, halo, nitro, C₁₋₄ alkyl, C₁₋₄ haloalkyl, —O(C₁₋₄ alkyl), —O(C₁₋₄ haloalkyl), NH₂, NH(C₁₋₄ alkyl) and N(C₁₋₄ alkyl)₂,
or a pharmaceutically or veterinarily acceptable salt or hydrate thereof, provided that the compound is not kinetin riboside.

Preferred features relating to R¹, R², R³, R⁴, R⁵ and X are as described above for the first aspect of the invention.

The compounds of General Formula (I) may be prepared by the methods disclosed below.

In particular, N⁶-substituted adenosine analogues of General Formula (I) may be prepared via a bimolecular nucleophilic substitution (S_{N2}) reaction by coupling the nucleoside derivative of a 6-halopurine with an amine nucleophile.

In such reactions, compounds of General Formula (I) are prepared via a single step process by reacting a 6-halopurine derivative of General Formula (V) with a nucleophile compound of General Formula (VI):

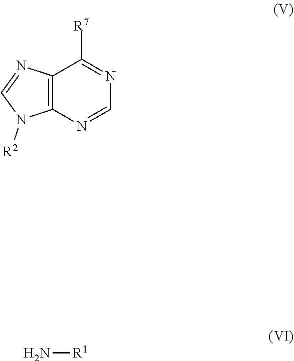

wherein R₇ is halo, preferably chloro, and R₁ and R₂ are as defined for General Formula (I). In preferred processes, this nucleophilic substitution reaction is carried out in the presence of a tertiary amine, preferably triethylamine (‘TEA’) and/or at elevated temperature (i.e. 30° C. or more).

Alternatively, the N⁶-substituted adenosine analogues of General Formula (I) may be prepared by reacting the nucleoside derivative of hypoxanthine with an amine nucleophile in the presence of a peptide coupling reagent.

In such reactions, compounds of General Formula (I) are prepared via a single step process by reacting a hyphoxanthine derivative of General Formula (VII) with a nucleophile compound of General Formula (VI) in the presence of benzotriazol-1-yloxytripyrrolidinophosphonium hexafluorophosphate (‘PyBOP’):

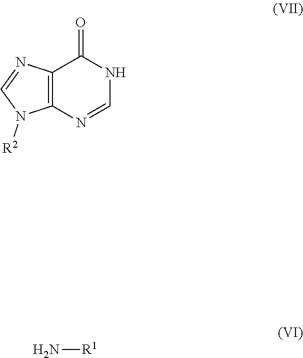

wherein R¹ and R² are as defined for General Formula (I). In preferred processes, this peptide coupling reaction is carried out in the presence of a tertiary amine, preferably N,N-Diisopropylethylamine (‘DIPEA’).

These methods for the preparation of a compound of General Formula (I) represent the third aspect of the invention.

Many compounds of General Formulae (V), (VI) and (VII) are well known and readily available. Other compounds of General Formulae (V), (VI) and (VII) can readily be synthesised by a person of skill in the art using standard methods.

According to a fourth aspect, the invention provides the use of kinetin riboside or a compound according to the second aspect of the invention in the preparation of an agent for the treatment of cancer or a disorder or condition that is associated with elevated levels of phosphorylated ubiquitin.

According to a fifth aspect, the invention extends to a method for treating cancer or a disorder or condition that is associated with elevated levels of phosphorylated ubiquitin, said method comprising administering to a patient in need of such a treatment an effective amount of kinetin riboside or a compound according to the second aspect of the present invention.

In preferred embodiments of the fourth and fifth aspects, the disorder or condition is selected from: idiopathic (i.e. aged and/or sporadic) Parkinson's disease, Lewy body dementia, and/or cancer. More preferably, the disorder or condition is selected from idiopathic Parkinson's disease and/or Lewy body dementia.

It will be appreciated that the kinetin riboside and/or the compounds of the second aspect of the invention will usually be administered as part of a pharmaceutical composition. Therefore, in a sixth aspect of the invention there is provided a pharmaceutical composition comprising kinetin riboside or a compound according to the second aspect of the invention and a pharmaceutically or veterinarily acceptable excipient or carrier.

Suitable pharmaceutical excipients are well known to those of skill in the art. Pharmaceutical compositions may be formulated for administration by any suitable route, for example oral, rectal, nasal, bronchial (inhaled), topical (including eye drops, buccal and sublingual), vaginal or parenteral (including subcutaneous, intramuscular, intravenous and intradermal) administration and may be prepared by any methods well known in the art of pharmacy.

The composition may be prepared by bringing into association kinetin riboside or a Compound of the second aspect of the invention with the carrier. In general, the formulations are prepared by uniformly and intimately bringing into association said compound with liquid carriers or finely divided solid carriers or both, and then if necessary shaping the product.

Formulations for oral administration in the present invention may be presented as: discrete units such as capsules, sachets or tablets each containing a predetermined amount of the compound; as a powder or granules; as a solution or a suspension of the compound in an aqueous liquid or a non-aqueous liquid; or as an oil-in-water liquid emulsion or a water-in-oil liquid emulsion; or as a bolus etc.

For compositions for oral administration (e.g. tablets and capsules), the term “acceptable carrier” includes vehicles such as common excipients e.g. binding agents, for example syrup, acacia, gelatin, sorbitol, tragacanth, polyvinylpyrrolidone (Povidone), methylcellulose, ethylcellulose, sodium carboxymethylcellulose, hydroxypropylmethylcellulose, sucrose and starch; fillers and carriers, for example corn starch, gelatin, lactose, sucrose, microcrystalline cellulose, kaolin, mannitol, dicalcium phosphate, sodium chloride and alginic acid; and lubricants such as magnesium stearate, sodium stearate and other metallic stearates, glycerol stearate, stearic acid, silicone fluid, talc waxes, oils and colloidal silica. Flavouring agents such as peppermint, oil of wintergreen, cherry flavouring and the like can also be used. It may be desirable to add a colouring agent to make the dosage form readily identifiable. Tablets may also be coated by methods well known in the art.

A tablet may be made by compression or moulding, optionally with one or more accessory ingredients. Compressed tablets may be prepared by compressing in a suitable machine the compound in a free-flowing form such as a powder or granules, optionally mixed with a binder, lubricant, inert diluent, preservative, surface-active or dispersing agent. Moulded tablets may be made by moulding in a suitable machine a mixture of the powdered compound moistened with an inert liquid diluent. The tablets may optionally be coated or scored and may be formulated so as to provide slow or controlled release of the active agent.

Other formulations suitable for oral administration include lozenges comprising the active agent in a flavoured base, usually sucrose and acacia or tragacanth; pastilles comprising the active agent in an inert base such as gelatin and glycerin, or sucrose and acacia; and mouthwashes comprising the active agent in a suitable liquid carrier.

Parenteral formulations will generally be sterile.

For topical application to the skin, the composition may be made up into a cream, ointment, jelly, solution or suspension etc. Cream or ointment formulations that may be used for the drug are conventional formulations well known in the art, for example, as described in standard textbooks of pharmaceutics such as the British Pharmacopoeia.

In a preferred embodiment of this aspect of the invention the composition is formulated for oral delivery.

The precise amount of a composition as defined herein which is therapeutically effective, and the route by which such compound is best administered, is readily determined by one of ordinary skill in the art. Such amounts will depend, of course, on the particular condition being treated, the severity of the condition, the individual patient parameters including age, physical condition, size and weight, the duration of the treatment, the nature of concurrent therapy (if any), the specific route of administration and like factors within the knowledge and expertise of the health practitioner. These factors are well known to those of ordinary skill in the art and can be addressed with no more than routine experimentation. It is generally preferred that a maximum dose of the individual components or combinations thereof be used, that is, the highest safe dose according to sound medical judgment. It will be understood by those of ordinary skill in the art, however, that a patient may insist upon a lower dose or tolerable dose for medical reasons, psychological reasons or for virtually any other reasons.

The doses of the compound or composition according to the invention administered to a subject can be chosen in accordance with different parameters, in particular in accordance with the mode of administration used and the state of the subject. Other factors include the desired period of treatment. In the event that a response in a subject is insufficient at the initial doses applied, higher doses (or effectively higher doses by a different, more localized delivery route) may be employed to the extent that patient tolerance permits.

In some cases, the compound of General Formula (I) may be used in combination with a further therapeutic agent, in particular a therapeutic agent used in the treatment of cancer or a neurodegenerative disease or condition that is characterised by an elevated level of Ubiquitin Ser65 phosphorylation.

Therefore in a seventh aspect of the invention, there is provided a combination therapeutic comprising a compound of General Formula (I) and an additional therapeutic agent used in the treatment of cancer or a neurodegenerative disease or condition, for simultaneous, separate or sequential use in the treatment of cancer or a neurodegenerative disease or condition that is characterised by an elevated level of Ubiquitin Ser65 phosphorylation.

When the compound of General Formula (I) and the additional therapeutic agent are used simultaneously, they may be provided in a pharmaceutical composition and therefore the invention also provides a pharmaceutical composition comprising a compound of General Formula (I), an additional therapeutic agent used in the treatment of a neurodegenerative disease or condition, and a pharmaceutically acceptable excipient or carrier for use in the treatment of a neurodegenerative disease or condition that is characterised by an elevated level of Ubiquitin Ser65 phosphorylation.

In some cases, the neurodegenerative disease or condition is idiopathic (i.e. aged or sporadic) Parkinson's disease, and the further therapeutic agent is an agent for treating Parkinson's disease. Examples of therapeutic agents used to treat Parkinson's Disease include Levodopa, ropinirole, rotigotine, pramipexole and amantadine, as well as folic acid or deoxynucleosides and their monophosphates.

Throughout the description and claims of this specification, the words “comprise” and “contain” and variations of the words, for example “comprising” and “comprises”, mean “including but not limited to” and do not exclude other moieties, additives, components, integers or steps. Throughout the description and claims of this specification, the singular encompasses the plural unless the context otherwise requires. In particular, where the indefinite article is used, the specification is to be understood as contemplating plurality as well as singularity, unless the context requires otherwise.

All references, including any patent or patent application, cited in this specification are hereby incorporated by reference. No admission is made that any reference constitutes prior art. Further, no admission is made that any of the prior art constitutes part of the common general knowledge in the art.

Preferred features of each aspect of the invention may be as described in connection with any of the other aspects.

Other features of the present invention will become apparent from the following examples. Generally speaking, the invention extends to any novel one, or any novel combination, of the features disclosed in this specification (including the accompanying claims and drawings). Thus, features, integers, characteristics, compounds or chemical moieties described in conjunction with a particular aspect, embodiment or example of the invention are to be understood to be applicable to any other aspect, embodiment or example described herein, unless incompatible therewith.

Moreover, unless stated otherwise, any feature disclosed herein may be replaced by an alternative feature serving the same or a similar purpose.

The Invention will now be described by way of example only with reference to the Examples below and to the following Figures wherein:

FIG. 1. Kinetin riboside suppresses niclosamide- and CCCP-induced Ub Ser65 phosphorylation. (A) A schematic representation of PINK1/Parkin signalling in healthy and damaged mitochondria. (B) Chemical structure of kinetin and its metabolism to generate the PINK1 ATP-neosubstrate, kinetin riboside triphosphate. (C) HeLa cells transfected with parkin were pre-treated with 50 μM kinetin riboside for 24 h and then they were either lysed or treated with 10 μM CCCP for 3 h. The cell lysates were probed for Ub Ser65 phosphorylation, OPA1 and GAPDH. UU: untreated and untransfected; UT: untreated and transfected. (D) HeLa cells transfected with YFP-parkin were pre-treated with 50 μM kinetin riboside for 24 h and then they were either lysed or treated with 10 μM niclosamide or CCCP for 1 h. The cell lysates were probed for Ub Ser65 phosphorylation, LC3B-I/-II and α-tubulin. N.T.: Non-treated cells. (E) As in (d), but samples studied using immunofluorescence for Ub Ser65 phosphorylation, YFP-parkin expression, and TOM20. Scale bar=40 μm.

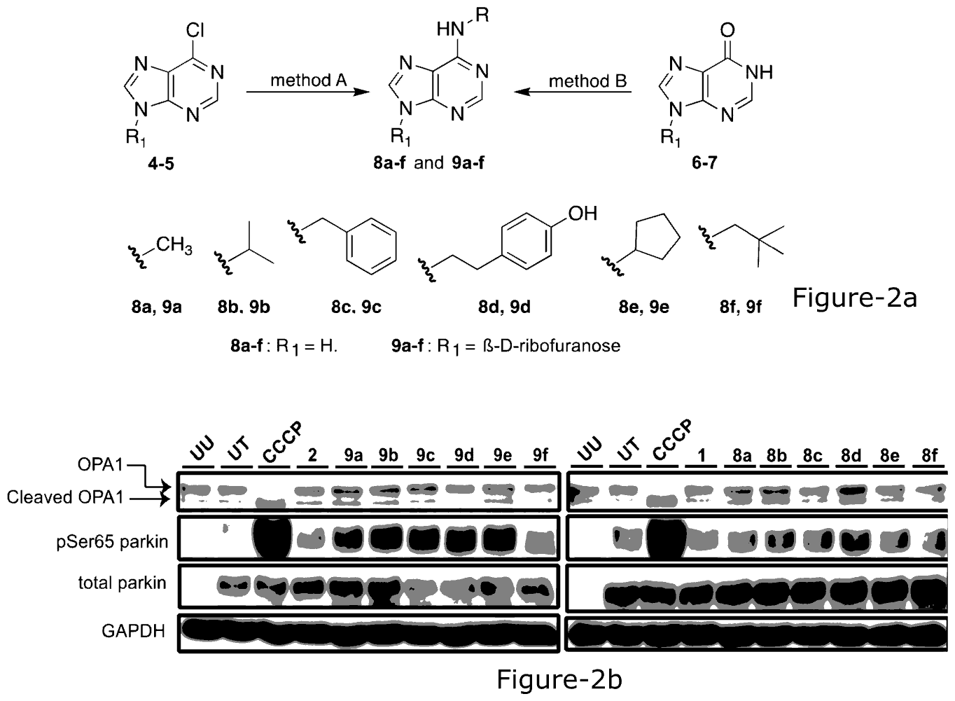

FIG. 2. Chemical synthesis N6-substituted adenines and adenosines and their activation of PINK1 in cells. (A) Method A reagents and conditions: triethylamine, ethanol, heating, 16 h. Method B reagents and conditions: PyBOP, DIPEA, acetonitrile/DMF, 3 days, rt. (B) HeLa cells transfected with Parkin were treated with 50 μM 1, 2, 8a-8f and 9a-9f for 1 h. CCCP was used at 10 μM and the treatment was for 3 h. Cells were then lysed and probed with anti-phospho Ser65 Parkin (pS65 Parkin), total parkin, OPA1 and GAPDH. UU: untreated and untransfected HeLa cells. UT: untreated and Parkin-transfected HeLa cells. The data is representative of three repeats.

FIG. 1. N⁶-benzyladenosine induces mitophagy and inhibits Ub Ser65 phosphorylation in cells and astrocytes. (A) HeLa cells transfected with YFP-parkin were pre-treated with 50 μM kinetin riboside or N⁶-benzyladenosine for 24 h and then they were either lysed or treated with 10 μM CCCP for 1 h. Immunofluorescence data probing for Ub Ser65 phosphorylation, YFP-parkin expression, and TOM20 of the samples. Scale bar=40 μm. (B) Quantification of Ub pSer65 localization to the mitochondria. (C) Astrocytes treated with 50 μM kinetin (1), N⁶-methyladenosine (9a), and N⁶-benzyladenosine (9c) followed by 10 nM valinomycin for 5 h. The samples were probed for phospho-Ub. (D) Induction of mitophagy in MEFs expressing PINK1 wild-type and PINK1 knock-out MEFs after treatment with 5 μM CCCP, N⁶-benzyladenosine (9c) and kinetin riboside (KR) for xx h. n=3. e. Quantification of MEFs undergoing mitophagy from d.

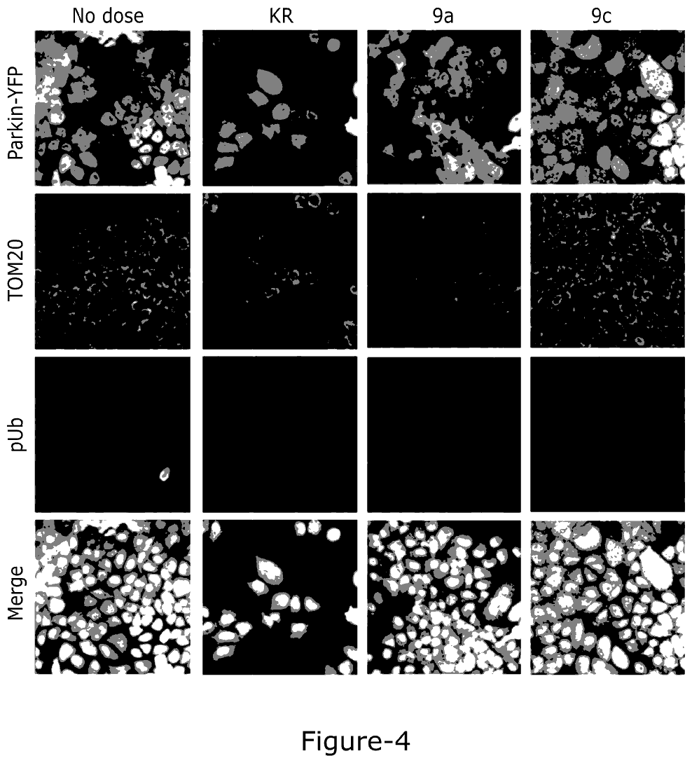

FIG. 4. Kinetin Riboside and nucleosides 9a and 9c do not impact mitochondrial fragmentation, parkin localization or ubiquitin phosphorylation.

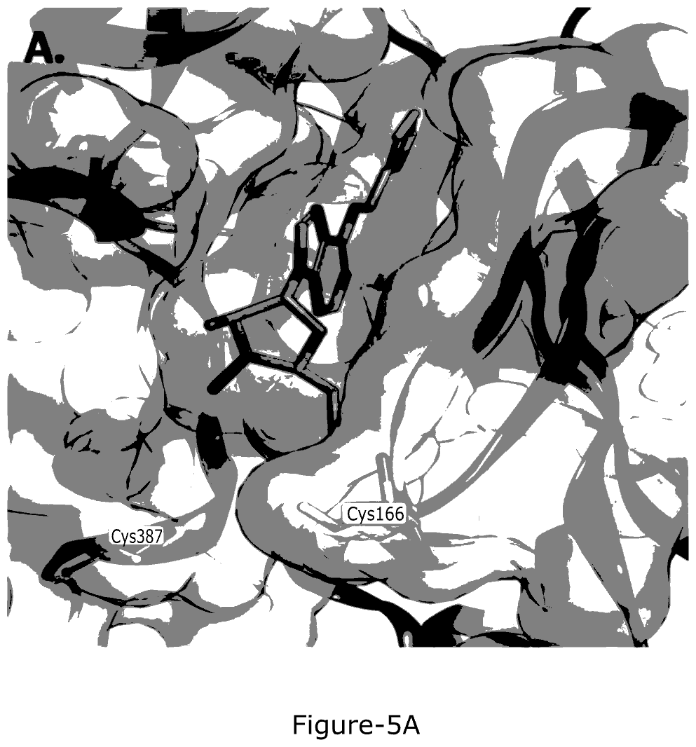

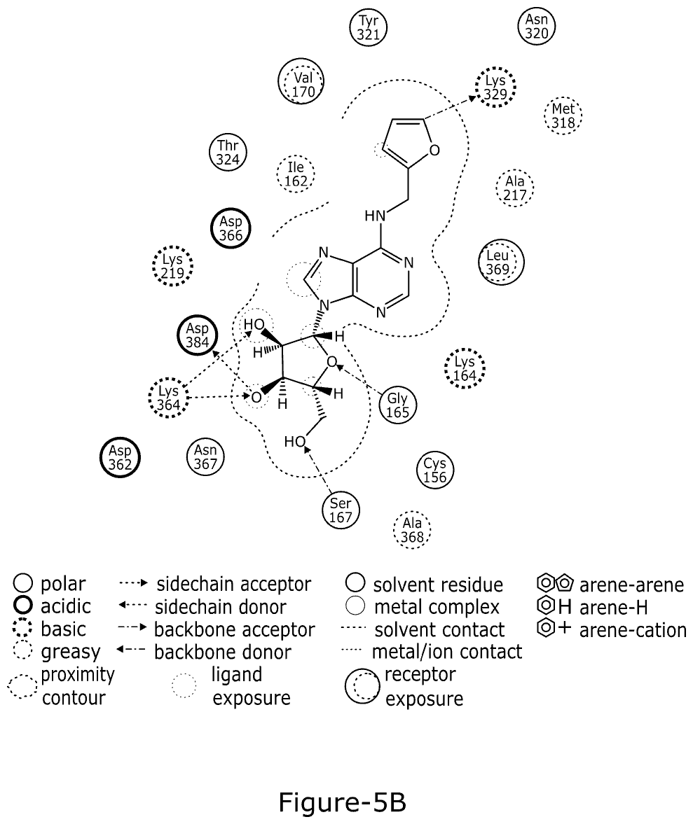

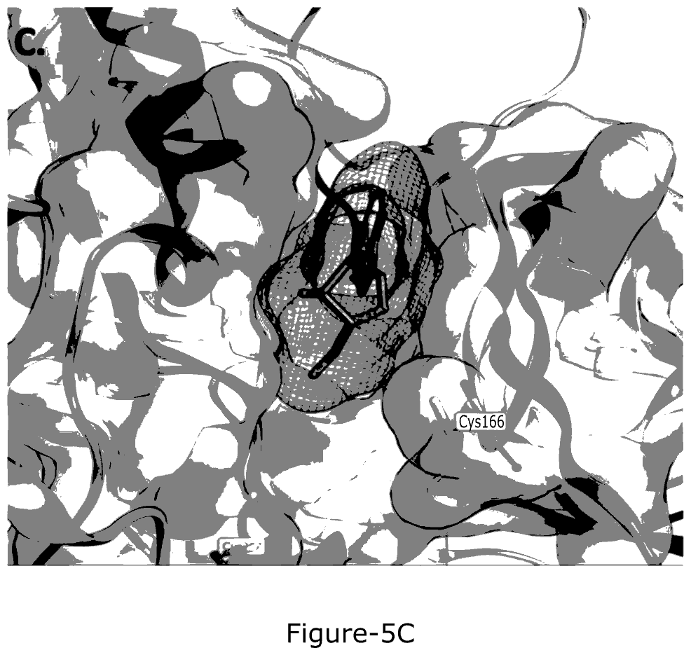

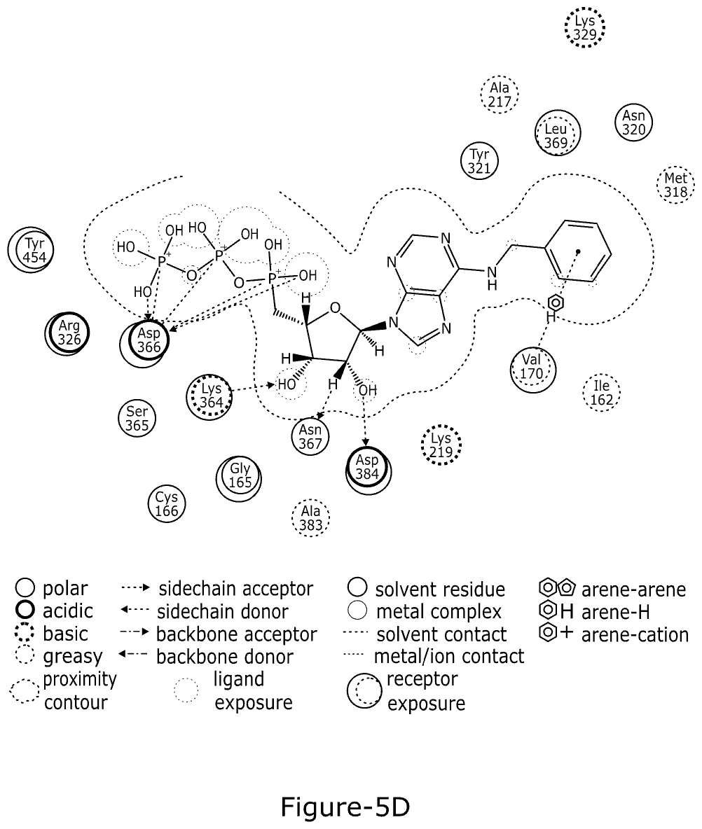

FIG. 5. Docking of kinetin riboside and nucleoside 9c into the human AlphaFold structure of human PINK1. (A) Docking of kinetin riboside into human PINK1. (B) 2D interactions between kinetic riboside and human PINK1. (C) Docking of nucleoside 9c into human PINK1. 2D interactions between nucleoside 9c and human PINK1.

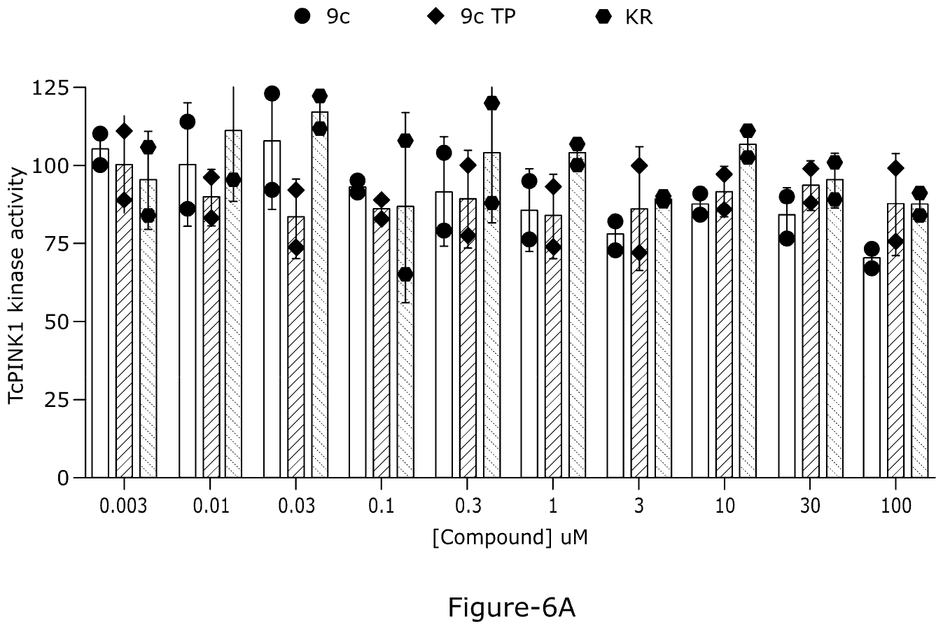

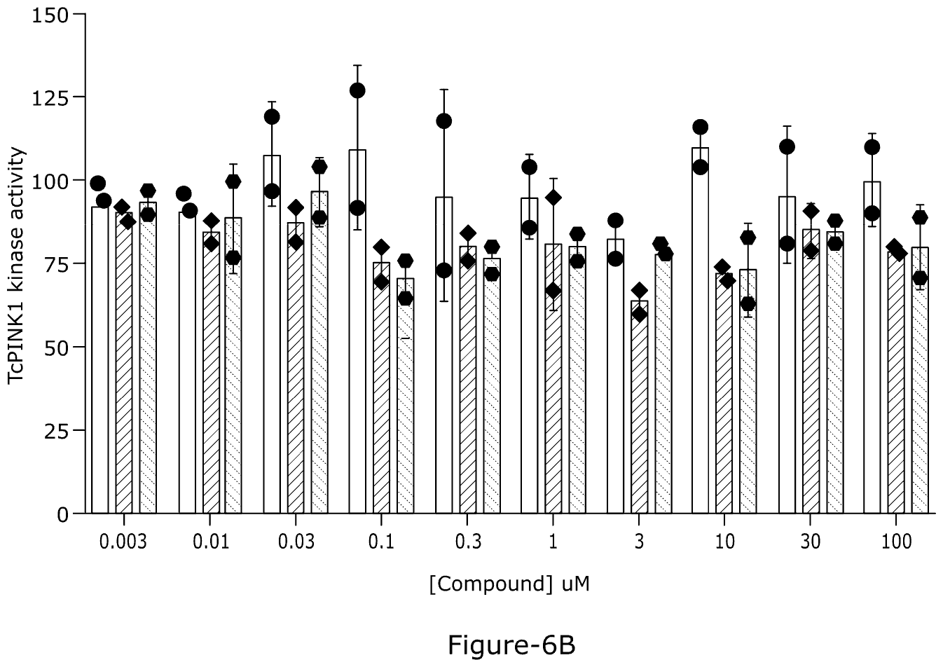

FIG. 6. Effect of kinetin riboside, nucleoside 9c, and its triphosphate derivative on TcPINK1 in vitro employing either ubiquitin (A) or parkin (B) as substrates.

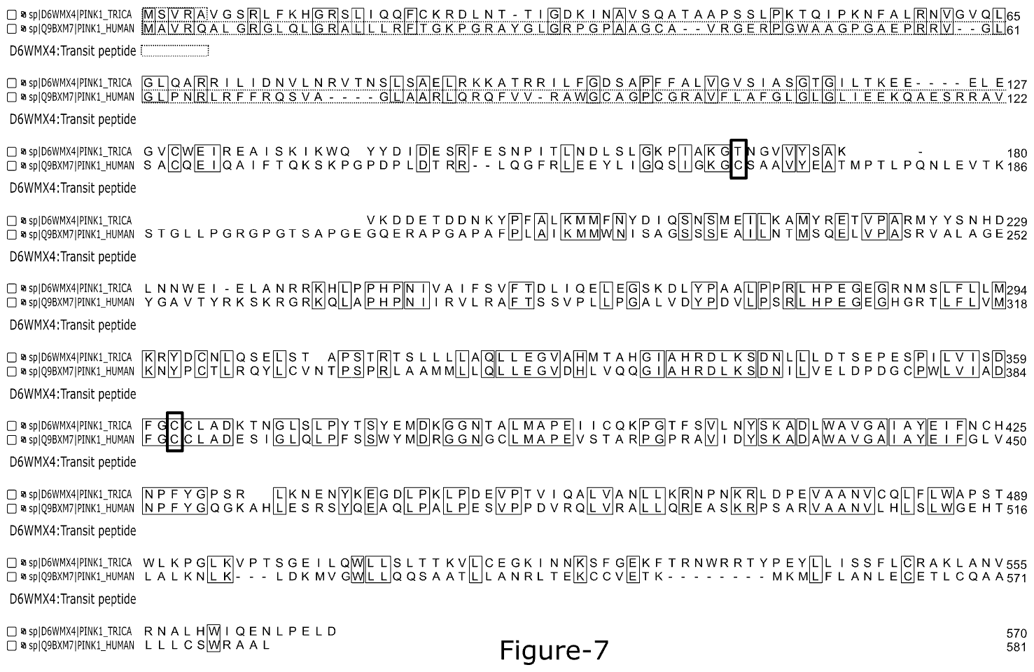

FIG. 7. Protein sequence alignment of human PINK1 (hPINK1; UniProt ID Q9BXM7) and Tribolium castaneum PINK1 (TcPINK1; UniProt ID D6WMX4). The data was obtained using UniProt Align. Human PINK1 Cyc166 (equivalent to TcPINK1 T172) and Cys387 (equivalent to TcPINK1 Cys362) are identified in red rectangles. The data shows hPINK1 Cyc166 not be conserved while hPINK1 Cyc387 is conserved.

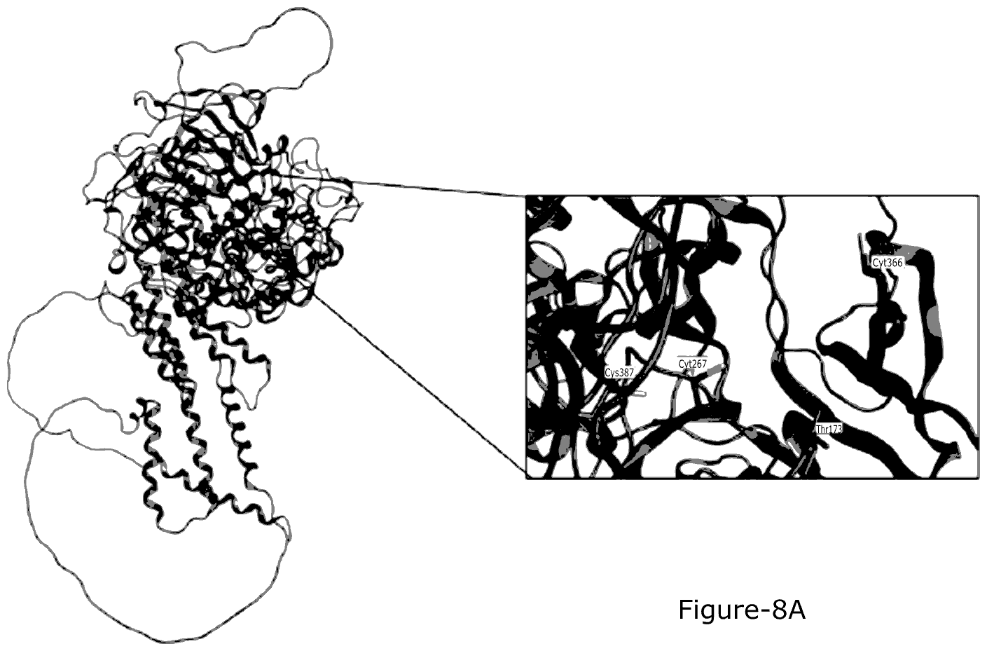

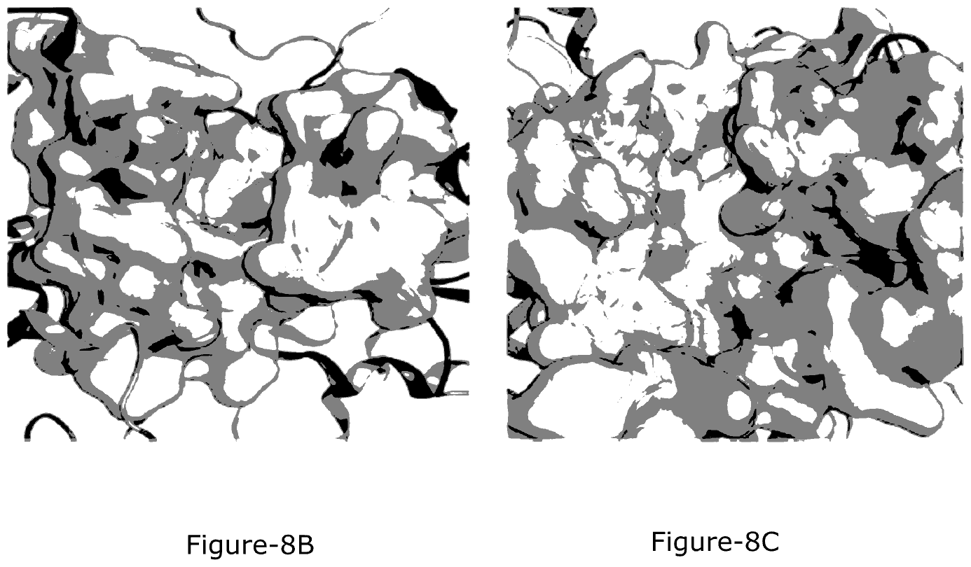

FIG. 8. (A) Superimposition of the full length AlphaFold human PINK1 (hPINK1, shown in red; UniProt ID Q9BXM7) and the full length Tribolium castaneum PINK1 (TcPINK1, shown in blue; UniProt ID D6WMX4). ATP binding pocket of (B) hPINK1 and (C) TcPINK1.

### MATERIALS AND METHODS

All reagents and solvents were of general purpose or analytical grade and were purchased from Sigma-Aldrich Ltd. (Merck), Fisher Scientific, Fluorochem, or Acros. 1H, and ¹³C NMR data were recorded on a Bruker AVANCE DPX500 spectrometer operating at 202, 500, and 125 MHz, respectively. Chemical shifts (6) are quoted in ppm, and J values are quoted in Hz. In reporting spectral data, the following abbreviations were used: s (singlet), d (doublet), t (triplet), q (quartet), dd (doublet of doublets), td (triplet of doublets), and m (multiplet). All of the reactions were carried out under a nitrogen atmosphere and were monitored using analytical thin layer chromatography on precoated silica plates (kiesel gel 60 F254, BDH). Compounds were visualized by illumination under UV light (254 nm) or by the use of KMnO4 stain followed by heating. Flash column chromatography was performed with silica gel 60 (230-400 mesh) (Merck). HPLC was carried out on a SHIMADZU Prominence-i quaternary low-pressure gradient pump with a Prominence-i UV detector (190 to 700 nm). All solvents for HPLC were HPLC grade purchased from Fisher Scientific. HPLC data analysis was performed using the SHIMADZU Lab solutions software package. The purity of the tested prodrugs was determined by HPLC, and they were all of >95% purity, except where specified.

### Cell Culture

Both HeLa and parkin-overexpressed HeLa cells were maintained in DMEM high glucose (Gibco), 10% FBS (Sigma-Aldrich) at 37° C. with 5% CO₂. For experiments, cells were counted and seeded into a range of culture plates, depending on the experiment, cell counting was done using the Cellometer Auto T4 with TrypanBlue (Gibco). HeLa cells were incubated at 37° C. in 5% CO2 in a T75 flask (Corning) until a confluency of 70-80% was achieved at which point the cells were used in an experiment. Alternatively, HeLa cells were seeded in well plates and then transfected with 0.2 pg ml-1 of parkin cDNA using the PEI method once the plated cells reached 60% confluency. After 6 hours, the media was changed. Sub-culturing was done when the cells reached ˜90% confluency.

### Preparation of Total Protein Lysate and Protein Concentration Measurement

Cell culture with 70-80% confluency was lysed as follows: cells were washed with Phosphate Buffer Saline (PBS) (Sigma). 150 μl of lysis buffer was used on each well containing either:
50 mM Tris-HCl pH 7.5, 1 mM EDTA, 1 mM EGTA, 0.27 M sucrose, 1 mM Na₃VO₄, 50 mM NaF, 5 mM Na pyrophosphate and fresh 1 mM benzamidine, 1% NP-40 and 0.1; or
50 mM Tris-HCl pH 7.5, 1 mM EDTA, 1 mM EGTA, 10 mM Na β-glycerophosphate, 0.27 M sucrose, 1 mM Na₃VO₄, 50 mM NaF, 10 mM Na pyrophosphate with fresh 1 mM benzamidine, 1% Triton X-100, complete EDTA-free Protease Inhibitor, Phosphatase inhibitor cocktail 3 and 100 μM of 2-chloroacetamide.

The cells were scrapped and transferred to the microtube and eventually spun down at 12,000 rpm for 15 minutes at 4° C. Finally, the supernatant was transferred to the new microtubes and stored at −20° C. Protein concentration was measured using Bradford Assay. Serial concentrations 0.125; 0.25; 0.5 and 1 mg/ml of Bovine Serum Albumin (BSA) (Sigma Aldrich) were used as a standard. Samples were boiled at 90° C. for 5 minutes in SDS sample loading buffer.

### Antibodies

Anti-GAPDH (1:1,000 5% BSA/TBS-T, Cell Signaling), Anti-PARKIN Phospho Ser65 (2 μg/ml, 5% milk/TBS-T, S210D, second bleed, University of Dundee), Anti-PARKIN Phospho Ser65 (2 μg/ml, 5% milk/TBS-T, S210D, third bleed, University of Dundee), Anti-PARKIN total (2 μg/ml, 5% milk/TBS-T, S966C, second bleed, University of Dundee), Anti-PARKIN Phospho Ser65 (1:10,000 in 5% BSA/TBS-T, rabbit monoclonal, MJF foundation), Non-phosphopeptide PARKIN Ser65 (2 mg/ml, 5% milk/TBS-T, University of Dundee), Anti-PINK1 total (2 μg/ml, 5% milk/TBS-T, S085D, third bleed, University of Dundee), Anti-Bcl-xL total (1:1,000, 5% BSA/TBS-T, Cell Signaling), Anti-Bcl-xL Phospho Ser62 (1:1,000, 5% BSA/TBS-T, Invitrogen), Anti-PINK1 Phospho Thr257 (2 μg/ml, 5% milk/TBS-T, S114D, third bleed, University of Dundee), Non-phosphopeptide PINK1 Thr257 (2 mg/ml, 5% milk/TBS-T, University of Dundee), Anti-OPA1 (1:1,000, 5% BSA/TBS-T, BD Biosciences). Anti-rabbit IgG HRP-linked (1:1,000 5% BSA/TBS-T, Cell Signaling), Anti-sheep IgG HRP-linked (1:5,000 5% milk/TBS-T, abcam), Anti-mouse IgG HRP-linked (1:1,000 5% BSA/TBS-T, Cell Signaling).

### Phospho-Ub Immunoblotting

Cells were pre-treated with kinetin riboside for 23h followed by 1 h treatment of 10 μM of CCCP or niclosamide. Cells were then harvested using AP lysis buffer (50 mM Tris-HCl (pH 7.5), 50 mM NaCl, 1% IGEPAL, 20 mM MgCl2, 5 mM 2-mercaptoethanol, 10% glycerol (v/v), 1× protease inhibitor cocktail (Roche), 1× phosphatase inhibitor cocktail (Roche). Denatured protein was loaded in Mini-PROTEAN TGX 4-12% precast gels, followed by protein separation. Proteins were then transferred to a methanol-activated PVDF membrane using Trans-Blot Turbo System (Bio-rad), membranes were subsequently blocked with 5% non-fat dry milk in TBS/0.1% tween (TBST) for 1 h at room temperature. Following blocking, membranes were washed in TBST and were incubated in primary antibodies diluted in 5% BSA/TBST or 5% milk/TSBT overnight at 4° C. with agitation. The next day membranes were washed in TBST and incubated with horse radish peroxidase (HRP)-conjugated antibodies for 1 h at room temperature with agitation. Following TBST washes, proteins were visualised using Amersham™ ECL™ Prime kit (GE Biosciences) and imaged using the Bio-rad ChemicDoc. Primary antibodies used for western blotting; GAPDH (Abcam cat #ab9485), Phospho-Ub Ser 65 (Sigma-Aldrich cat #ABS1513-1), LC3B (Cell Signaling cat #3868S). Secondary antibodies used for western blotting; anti-rabbit HRP (Agilent Dako cat #P0399), anti-mouse HRP (Agilent Dako cat #P0260).

### Immunofluorescence

Cells were seeded on PerkinElmer PhenoPlate 96-well plates, followed by treatment with compounds. Cells were fixed in chilled 4% PFA for 10 min followed by washes in PBS. Permeablisation of cells was achieved with the addition of PBS/0.1% Triton X-100 for 5 mins at room temperature. Following PBS washes, cells were blocked using blocking buffer (PBS, 5% BSA, 0.1% Tween20) for 1 h at room temperature. Primary antibodies were diluted in blocking buffer and added to cells to be incubated overnight at 4° C. Cells were subsequently washed with PBS-T and the appropriate secondary antibodies (diluted in blocking buffer) were added for 2 h at room temperature in darkness. 10 μg/ml of Hoechst 33258 stain was added with secondary antibodies when required. Cells were again washed with PBS-T followed by addition of PBS with 0.02% sodium azide. Cells were then stored at 4° C. until ready for imaging. Primary antibodies used for immunofluorescence are as follows: Phospho-Ub Ser 65 (Sigma-Aldrich cat #ABS1513-1), TOM20 (Santa-Cruz cat #sc-17764). Secondary antibodies and stains used in immunofluorescence; Anti-IgG2a Alexa Fluor™ 546 (ThermoFisher cat #A21133) Anti-rabbit Alexa Fluor™ 647 (ThermoFisher cat #A21244), Hoechst 33258 stain (Sigma-Aldrich cat #B2883).

Confocal microscopy was performed using a Zeiss LSM800 w/Airyscan fluorescent microscope equipped with lasers emitting at 405 nm, 488 nm, 561 nm and 640 nm. All images were captured using a 40x oil immersion objective lens and processed using Zeiss ZEN software. Images were analysed and quantified using Columbus (PerkinElmer) and TIBCO Spotfire software.

For assessing tetramethylrhodamine methyl ester perchlorate (TMRM) fluorescence, cell media was removed and replaced with cell media consisting of 5 nM TMRM (ThermoFisher) and 10 μg/ml Hoechst 33258 stain for 30 mins in the dark at 37° C. to allow incorporation of TMRM into mitochondria. Following PBS washes, FluoroBrite DMEM (Gibco) media was added to cells, followed by subsequent imaging using a Zeiss LSM800 microscope at 37° C. with 5% CO₂.

### PINK1 In Vitro Kinase Assay

This assay was carried out by the MRC PPU Reagents and Services (University of Dundee, U.K.). The assay was conducted as follows: TcPINK1 (5-20mU diluted in 50 mM Tris pH 7.5, 0.1 mM EGTA, 1 mg/ml BSA, 0.1% mercaptoethanol) is assayed against GST PARK2 TV3 or Ubiquitin-His in a final volume of 25.5 pl containing 50 mM Tris pH 7.5, 0.1 mM EDTA, 10 mM DTT, protein substrate (0.3 mg/ml of GST PARK2 TV3 or 1 mg/ml of Ubiquitin-His, 10 mM magnesium acetate and 0.1 mM [33P-γ-ATP](50-1000 cpm/pmole) in the presence of the relevant small molecule (serial dilution with the highest concentration being 100 μM) and incubated for 30 min at room temperature. Assays are stopped by addition of 5 μl of 0.5 M (3%) orthophosphoric acid and then harvested onto P81 Unifilter plates with a wash buffer of 50 mM orthophosphoric acid.

### Example 1: PINK 1 Activation (Kinetin Riboside)

We identified that the previously reported PINK1 activation by kinetin riboside 2 in cells was noted at a high concentration (50 μM), and detectable PINK1 activation in cells by the nucleobase kinetin was only observed in the presence of the mitochondrial uncoupler CCCP. Therefore, we initially asked whether combining this nucleoside analogue, kinetin riboside 2, with some of the known indirect PINK1 activators such as niclosamide and CCCP would lead to a synergistic and more significant activation of PINK1. To explore this, we first treated parkin-transfected HeLa cells for 24 h with 50 μM kinetin, kinetin riboside or kinetin riboside ProTide, a monophosphate prodrug of kinetin riboside. Subsequently, the cells were either lysed or treated with 10 μM CCCP for 3 h (FIG. 1c). Niclosamide and CCCP treatments alone were used as controls. Upon cell lysis and probing for Ub Ser65 (pUb), optic atrophy protein 1 (OPA1) and GAPDH, as expected niclosamide and CCCP treatments alone produced strong phosphorylation of Ub at Ser65 (FIG. 1c). The treatment of cells with 50 μM kinetin, kinetin riboside or kinetin riboside ProTide alone did not lead to any notable phosphorylation of ubiquitin. Strikingly, the pre-treatment of cells with kinetin riboside inhibited the CCCP-induced phosphorylation of ubiquitin while the related nucleobase kinetin or kinetin riboside ProTide pre-treatment did not. The treatment of cells with niclosamide and CCCP alone led to OPA1 cleavage indicating mitochondrial membrane depolarization, while pre-treatment with kinetin riboside did not prevent this effect (FIG. 1c).

Surprised by this finding, we next explored if this same inhibition of CCCP-induced ubiquitin phosphorylation by kinetin riboside can also be observed with the mitochondrial uncoupler niclosamide. For this, HeLa cells transfected with YFP-parkin were pre-treated with 50 μM kinetin riboside for 24 h and then the cells were either lysed or treated with 10 μM niclosamide or CCCP for 1 h. Again, and as expected, the control samples in which the cells were treated with only niclosamide and CCCP induced strong ubiquitin phosphorylation whereas the pre-treatment of cells with kinetin riboside inhibited both the niclosamide- and CCCP-induced phosphorylation of ubiquitin (FIG. 1d) similar to the outcome in FIG. 1c.

The ability of kinetin riboside to inhibit ubiquitin phosphorylation was then probed using immuno-fluorescence (FIG. 1e). YFP-parkin transfected HeLa cells were either left untreated or treated with 10 μM niclosamide or 10 μM CCCP alone for 1 h or pre-treated with 50 μM kinetin riboside for 24 h prior to the addition of niclosamide or CCCP as illustrated in FIG. 1d. Treatment of cells with niclosamide and CCCP again induced significant phosphorylation of ubiquitin while pre-treatment of cells with kinetin riboside inhibited ubiquitin phosphorylation (FIG. 1e). Although pre-treatment with kinetin riboside inhibited ubiquitin phosphorylation, it did not prevent the membrane potential collapse caused by niclosamide and CCCP (not shown) akin to the observation noted by probing for OPA1 cleavage (FIG. 1c).

### Example 2: Synthesis of N⁶-Substituted Adenine and Adenosine Analogues

Intrigued by the ability of kinetin riboside to inhibit niclosamide- and CCCP-induced ubiquitin phosphorylation, we subsequently designed and synthesized N⁶-substituted adenines and adenosines that are structurally related to kinetin and kinetin riboside. In the design of these nucleobases and nucleosides, we elected to modify the N⁶-position of adenine and adenosine and chose to make various small, medium and bulky substitutions at this position. Precisely, the N⁶-substitutions were methyl, isopropyl, benzyl, tyramine, cyclopentylamine, neopentylamine and furfuryl (8a-f and 9a-f, FIG. 2a).

The synthesis of N⁶-substituted adenine and adenosine compounds was carried out using one of two methods depending on the volatility of the nucleophile being used in the reaction (FIG. 2a). Standard nucleophilic S_{N2} substitution was the preferred method (Method A) as it gave high yields after a simple purification step. This method involved heating 6-chloropurine or its nucleoriboside derivative with the corresponding nucleophile in ethanol in the presence of triethylamine. This method was employed in the synthesis of the adenine and adenosine analogues that had either a benzyl (8c and 9c), isopropyl (8d and 9d), methyl (8e and 9e) or tyramine (8f and 9f) modifications at the N⁶-position. In Method B, the peptide coupling agent PyBOP was used for the synthesis of nucleobases and nucleosides bearing cyclopentylamine and neopentylamine at the N⁶-position. The choice to employ method B for the synthesis using neo-pentylamine (8a and 9a) and cyclopentylamine (8b and 9b) as nucleophiles was driven by the fact that these compounds are explosive when heated, ruling out using Method A. For the synthesis of the nucleobases and nucleosides in this case, hypoxanthine or inosine was added to PyBOP and partially dissolved in mixture of acetonitrile and substoichiometric quantities of DMF. The corresponding nucleophiles, cyclopentylamine or neo-pentylamine, were then added and left to react for three days.

Compared to Method A, Method B gave a much lower yields and a more complicated purification of the final compounds.

Further details of the above syntheses can be found in the appendix.

### Example 3: PINK 1 Activation (N⁶-Substituted Adenine and Adenosine Compounds)

Upon completion of the syntheses of Example 2, the ability of the synthesized nucleobases and nucleosides to activate PINK1 in cells was assessed. HeLa cells, which express PINK1 endogenously, but not parkin, were transiently transfected with parkin. Subsequently, the cells underwent treatment with nucleobases (8a-8f) and nucleosides (9a-9f) for 1 h, or 10 μM CCCP for 3 h as a control, and probed for parkin Ser65 phosphorylation, total Parkin, OPA1 and GAPDH as a loading control. The results showed that CCCP resulted in prominent activation of PINK1 as judged by parkin Ser65 phosphorylation while nucleobases 8a-8f did not show any significant activation of PINK1 at 50 μM in agreement with previous findings (FIG. 2b). However, all of the nucleosides studied (9a-9f) exhibited pronounced activation of PINK1 apart from compound 9f (FIG. 2b). Interestingly, the activation of PINK1 by these nucleosides did not result in the cleavage of OPA1. This indicates that these nucleoside analogues activate PINK1, independently of mitochondrial depolarization, in contrast to CCCP activation of PINK1 which was associated with cleavage of OPA1. This is because this agent, CCCP, activates PINK1 indirectly via the depolarization of the mitochondrial membrane in line with previous observations.

### Example 4: CCCP/Niclosamide Independent Activation of PINK1

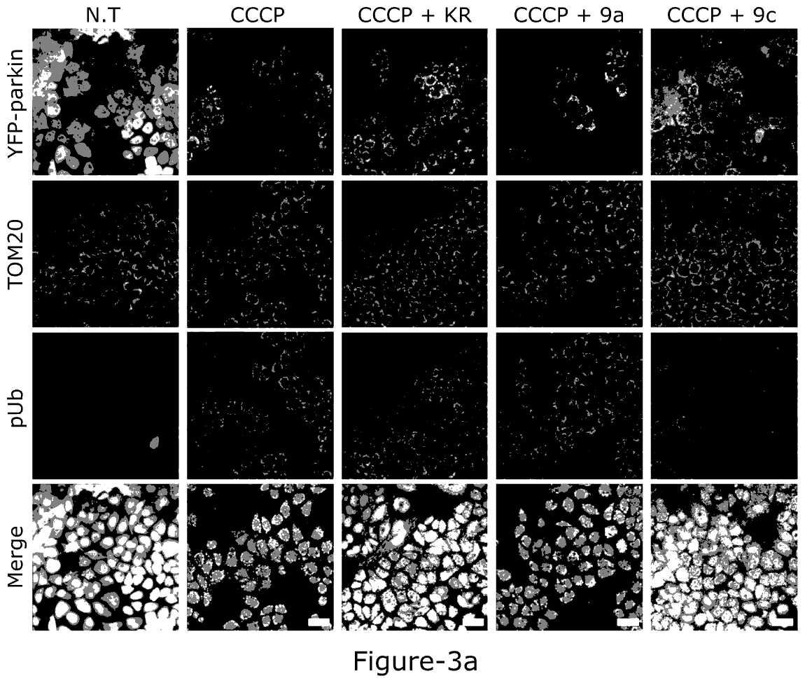

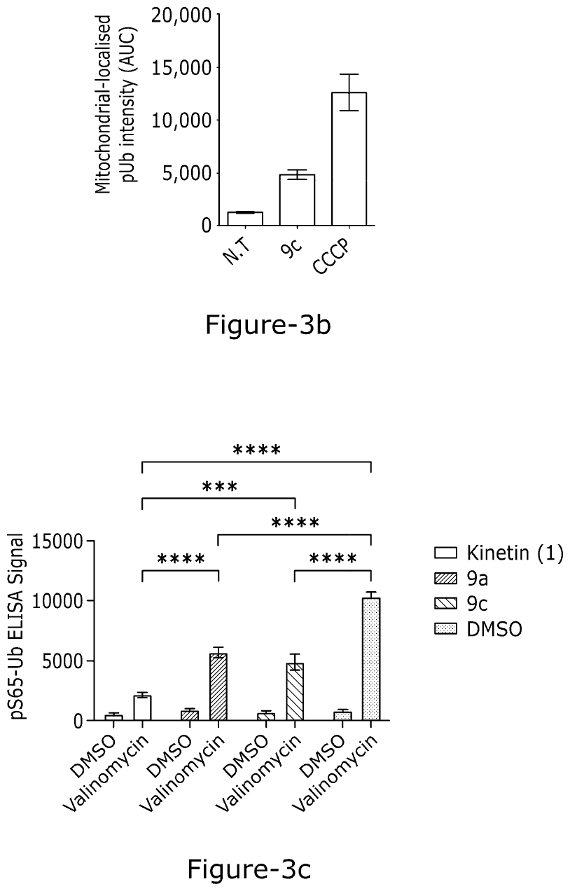

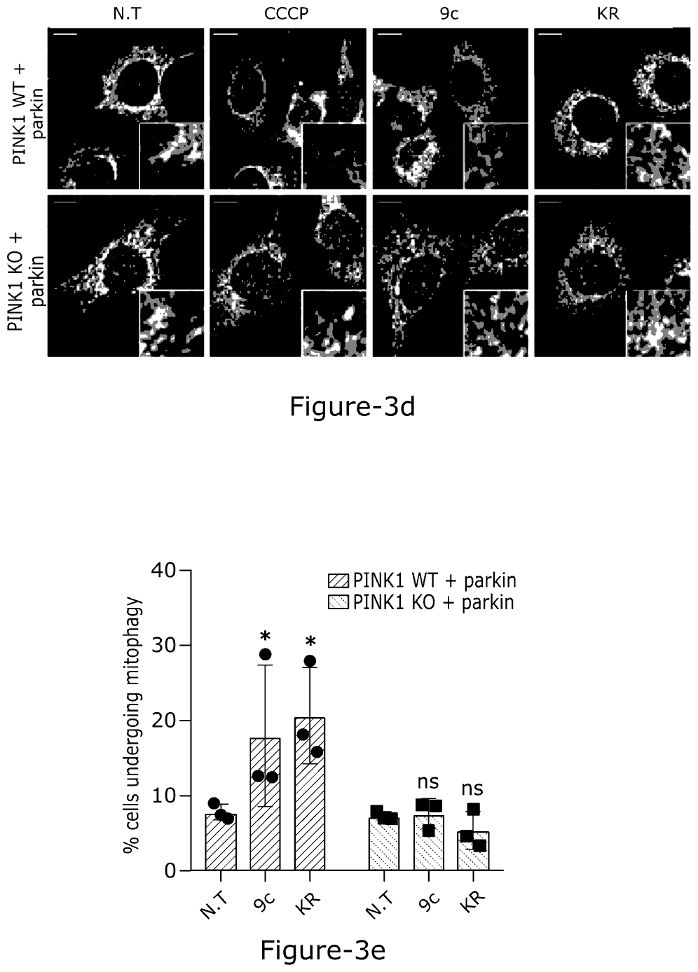

Next, we explored the impact of kinetin riboside, nucleosides 9a and 9c on mitochondria in the absence of niclosamide and CCCP. First, HeLa cells transfected with YFP-parkin were either left untreated or treated with 50 μM kinetin riboside, nucleosides 9a and 9c for 24 h. Probing for TOM20 by immunofluorescence in these cells indicated that these nucleoside analogues alone did not have any impact on mitochondrial fragmentation, parkin localisation or ubiquitin phosphorylation (FIG. 3). Subsequently, YFP-parkin expressing HeLa cells were treated with 50 μM kinetin riboside, nucleosides 9a or 9c for 24 h, and this was followed by 10 μM CCCP treatment. Using immunofluorescence to monitor ubiquitin Ser65 phosphorylation, CCCP treatment induced strong ubiquitin phosphorylation and promoted parkin localisation to the mitochondria (FIG. 3a). Notably, ubiquitin phosphorylation was inhibited by kinetin riboside pre-treatment in agreement with the data (FIG. 2c-e). In terms of the new compounds, pre-treatment with nucleoside analogue 9a followed by CCCP treatment did not have a significant impact on ubiquitin phosphorylation and parkin localisation to the mitochondria compared to the CCCP treatment alone (FIG. 3a). However, pre-treatment with nucleoside analogue 9c produced significant reduction in ubiquitin phosphorylation and parkin localisation to the mitochondria, which appears stronger than that induced by kinetin riboside (FIGS. 3a and b).

With the consistent observation that kinetin riboside and nucleoside 9c inhibit ubiquitin phosphorylation in cells, we explored whether this effect was evident in astrocytes. Briefly, astrocytes were treated with 50 μM of the nucleobase kinetin, nucleosides 9a and 9c or DMSO for 24 h and 5 h prior to lysis, the samples were treated with 50 nM valinomycin, a mitochondrial uncoupler. Subsequently, probing for ubiquitin Ser65 phosphorylation indicated that treatment with the nucleobase kinetin, nucleosides 9a and 9c alone resulted in increased phosphorylation of ubiquitin Ser65 phosphorylation in line with the results observed with parkin Ser65 phosphorylation shown in FIG. 2b (FIG. 3c). Interestingly, the pre-treatment of astrocytes with kinetin, nucleosides 9a and 9c for 24 h followed by treatment with valinomycin for 5 h led to significant suppression of ubiquitin Ser65 phosphorylation as compared the treatment of astrocytes with valinomycin alone (black bar, FIG. 3c).

### Example 5: PINK1 Dependent Mitophagy

With the inhibition of ubiquitin phosphorylation and parkin localisation to the mitochondria by kinetin riboside and the nucleoside analogue 9c, we next asked whether these compounds still induce mitophagy in a PINK1 dependent manner. To explore this, we employed the established MitoQC assay for measuring mitophagy in cells^{11,12}. Indeed, immortalised MEF expressing either PINK1 wild-type (WT) or knock-out (KO) were treated with 20 μM CCCP or 5 μM of kinetin riboside or compound 9c for 24 h. CCCP treatment induced an increase in mitophagy in MEFs expressing PINK1 wild-type, but not in PINK1 KO MEFs, in line with previous reports (FIG. 3d-e. Enlarged FIG. 3d). Interestingly, the treatment of the PINK1 WT MEFs with kinetin riboside or the nucleoside analogue 9c also induced mitophagy while this was not observed in the PINK1 KO MEFs (FIG. 3d-e). This data indicates that both kinetin riboside and the nucleoside analogue 9c induce a low level of mitophagy in a PINK1-dependent manner, which is in line with the previous observation that kinetin riboside and nucleoside 9c (FIG. 2b) induce low level activation of PINK1 as judged by parkin phosphorylation.

### Example 6: Molecular Modelling Studies

Together, the results of Examples 1 to 5 showcase the ability of kinetin riboside and N⁶-substituted adenosine compounds, in particular N⁶-benzyladenosine (9c) to inhibit niclosamide- and CCCP-induced ubiquitin phosphorylation. To the best of our knowledge, there have been two examples of inhibiting PINK1's ability to phosphorylate ubiquitin^{13,14}. The first concerns PD-associated mutations C125G and Q126P that disrupt an intramolecular interaction between the human PINK1's N- and C-terminal extensions leading to the inhibition of ubiquitin phosphorylation¹⁴, while the second is the oxidation of human PINK1 Cys166 and/or Cyc387.¹³ Structural inspection of the AlphaFold human PINK1 structure and molecular modelling studies (FIG. 5) indicated that these cysteine residues are remote from the binding site of the nucleobase of the nucleoside analogues and their triphosphate derivatives and it is, therefore, unlikely that these can be covalent inhibitors.

To examine this further, we ran in vitro kinase assays employing recombinant and constitutively active Tribolium castaneum PINK1 (TcPINK1) and either human parkin or ubiquitin as substrates in the presence of increasing concentrations (max 100 μM) of kinetin riboside, benzyladenosine 9c or its triphosphate derivative (FIG. 6). Although the results suggest that these compounds are unlikely to be PINK1 covalent inhibitors, it must be noted that the human PINK1 Cyc166 is not conserved in TcPINK1 while Cyc387 is conserved across human PINK1 and TcPINK1 (FIG. 7). Additionally, the superimposition of the ATP pockets of the hPINK1 and TcPINK1 showed that the TcPINK1 ATP pocket to be more open than that of hPINK1, and although the position of hPINK1 Cyc387 was close to the TcPINK1 Cys362, the position of the TcPINK1 Thr172 appear far remote from that of hPINK1 Cyc166 (FIG. 8), which appears to have an impact of hPINK1's ability to phosphorylate ubiquitin. In terms of the second possibility of kinetin riboside and 9c disrupting the intramolecular interaction between PINK1's N- and C-terminal extensions akin to the Q126P mutation, this may result from the binding of these compounds or their phosphorylated species to the ATP pocket of PINK1 causing a conformational change that rearranges the N- and C-ends leading to the inability of PINK1 to phosphorylate ubiquitin.

### SUMMARY

We herein describe nucleoside analogues as inducers of low level PINK1-mitophagy, and which suppress ubiquitin phosphorylation caused by mitochondrial uncouplers, CCCP, valinomycin and niclosamide. These nucleoside analogues represent useful tool compounds for decoding the role of phosphoubiquitin signalling, including its parkin-independent functions such as the inhibition of E2/E3 ubiquitin systems and deubiquitinases. Critically, as post-mortem analysis of the substantia nigra of Lewy body dementia patients (a major site of neuronal loss) as well as aged PD patients revealed elevated levels of Ub Ser65 phosphorylation compared with healthy age-matched controls,⁹⁻¹⁰ nucleoside analogues that exhibit inhibition of ubiquitin phosphorylation could therefore have utility in treating Lewy body dementia or idiopathic PD.

### APPENDIX

### Synthesis and Evaluation of N⁶-Substituted Adenine and Adenosine Analogues

### N-methyl-9H-purin-6-amine (8a)

Methylamine (0.40 mL, 9.01 mmol, 2.8 eq.) and TEA (0.45 mL, 3.23 mmol, 1 eq.) were added to a stirring solution of 6-chloropurine (500 mg, 3.23 mmol, 1 eq.) in EtOH (15 mL). The reaction was then heated to 30° C. for 16 h. The solvent was then removed under reduced pressure. The crude oil that remained was then purified by column chromatography using DCI/MeOH (19:1) as an eluent to give a white solid (200 mg, 41%); ¹H NMR (500 MHz, DMSO) δ 12.93 (1H, s, NH), 8.25 (1H, s, H-4), 8.13 (1H, s, H-1), 7.60 (1H, s, NH), 3.02 (3H, s, 46); ¹³C NMR (126 MHz, DMSO) δ 152.90 (C-4), 152.85 (C-3), 27.40 (C-6); HRMS-ES (m/z): Found [M+H]⁺ [150.0781, [C₆H₇N₅H] requires 150.0780. HPLC(reverse phase) 0.5 mL/min MeOH/H₂O 80:20 in 12 min, λ=254 nm, Rt=4.88 min (99%).

### N-isopropyl-9H-purin-6-amine (8b)

Isopropylamine (0.28 mL 3.29 mmol, 1 eq.) and TEA (0.45 ml, 3.23 mmol, 1 eq.) were added to a stirring solution of 6-chloropurine (500 mg, 3.23 mmol, 1 eq.) in EtOH (15 mL). The reaction was then heated to 40° C. for 16 h. The solvent was then removed under reduced pressure. The crude oil that remained was then purified by column chromatography using DCM/MeOH (19:1) as an eluent to give a white solid (11.7 mg, 2%); ¹H NMR (500 MHz, MeOD) δ 8.21 (1H, s, H-4), 8.06 (1H, s, H-1), 3.25-303 (1H, m, H-6), 1.32 (6H, d, J=6.5 Hz, H-7); ¹³C NMR (126 MHz, MeOD) δ 46.52 (C-6), 7.82 (C-7). HPLC(reverse phase) 0.5 mL/min MeOH/H₂O 90:10 in 12 min, λ=254 nm, Rt=4.69 min (99%).

### N-benzyl-9H-purin-6-amine (8c)

Benzylamine (0.42 ml, 3.88 mmol, 1.2 eq.) and TEA (0.54 ml, 3.88 mmol, 1.2 eq.) were added to a stirring solution of 6-chloropurine (500 mg, 3.24 mmol, 1 eq.) in EtOH (15 mL). The reaction was then refluxed at 80° C. for 16 h. The product crashed out of solution when placed in an ice water bath and stirred vigorously. OL035 was then filtered off and further purified by column chromatography using DCM/MeOH (19:1) as an eluent to give a white solid (240 mg, 33%); ¹H NMR (500 MHz, MeOD) δ 8.25 (1H, s, H-1), 8.07 (1H, s, H-4), 7.39 (2H, d, J=7.6 Hz, H-8), 7.32 (2H, t, J=7.5 Hz, H-9), 7.25 (1H, t, J=7.3 Hz, H-10); ¹³C NMR (126 MHz, MeOD) δ 152.42 (C-4), 128.18 (C-9), 127.18 (C-8), 126.88 (C-10) 46.48 (C-6); HRMS-ES (m/z): Found [M+H]⁺ 226.1089, [C₁₂H₁₁N₅H] requires 226.1093. HPLC (reverse phase) 0.5 mL/min MeOH/H₂O 80:20 in 12 min, λ=254 nm, Rt=5.63 min (99%).

### 4-(2-((9H-purin-6-yl)amino)ethyl)phenol (8d)

Tyramine (533 mg, 3.88 mmol, 1.2 eq.) and TEA (0.54 mL, 3.88 mmol, 1.2 eq.) were added to a stirring solution of 6-chloropurine (500 mg, 3.24 mmol, 1 eq.) in EtOH (15 mL). The reaction was then refluxed at 80° C. for 16 h. The product crashed out of solution when placed in an ice water bath and stirred vigorously. OL037 was then filtered off and further purified by column chromatography using DCM/MeOH (19:1) as an eluent to give a white solid (141 mg, 17%); ¹H NMR (500 MHz, DMSO) δ 8.20 (1H, s, H-1), 8.07 (1H, s, H-4), 7.05 (2H, d, J=7.8 Hz, H-9), 6.69 (2H, d, J=8.5 Hz, H-10), 3.64-3.63 (2H, m, H-6) 2.81-2.78 (2H, m, H-7); ¹³C NMR (126 MHz, DMSO) δ 156.09 (C-11), 130.04 (C-8), 129.96 (C-9), 115.58 (C-10), 45.80 (C-6), 36.53 (C-7); HRMS-ES (m/z): Found [M+H]⁺ 256.1205, [C₁₃H₁₃N₅OH] requires 256.1198. HPLC(reverse phase) 0.5 mL/min MeOH/H₂O 80:20 in 12 min, λ=254 nm, Rt=4.96 min (86%).

### N-cyclopentyl-9H-purin-6-amine (8e)

Hypoxanthine (500 mg, 3.67 mmol, 1 eq.), cyclopentylamine (0.54 ml, 5.51 mmol, 1.5 eq.) and PyBOP (2.294 g, 4.41 mmol, 1.2 eq.) were dissolved in anhydrous ACN (20 mU and substoichiometric amounts of DMF (2 ml) under an inert atmosphere. DIPEA(1.28 ml, 7.35 mmol, 2 eq.) was then added slowly over 5 min at RT and the reaction was left to stir for 3 days. The solvent was then removed under reduced pressure. The crude oil that remained was then purified by column chromatography using DCM/MeOH (19:1) as an eluent to give a white solid (130 mg, 17%); ¹H NMR (500 MHz, MeOD) δ 8.23 (1H, s, H-4), 8.07 (1H, s, H-1), 2.14-2.07 (1H, m, H-6), 1.85-1.60 (8H, m, H-7,8); ¹³C NMR (126 MHz, DMSO) δ 152.80 (C-4), 32.77 (C-6), 23.91 (C-7,8). HRMS-ES (m/z): Found [M+H]⁺ 204.1244, [C₁₀H₁₃N₅H] requires 204.1249. HPLC (reverse phase) 0.5 mL/min MeOH/H₂O 80:20 in 12 min, λ=254 nm, Rt=5.78 min (99%).

### N-neopentyl-9H-purin-6-amine (8f)

Hypoxanthine (300 mg, 2.20 mmol, 1 eq.), neopentylamine (0.39 mL, 3.31 mmol, 1.5 eq.) and PyBOP (1.721 g, 3.31 mmol, 1.5 eq.) were dissolved in anhydrous ACN (20 mL) and substoichiometric amounts of DMF (2 ml) under an inert atmosphere. DIPEA (0.77 ml, 4.41 mmol, 2 eq.) was then added slowly over 5 min at RT and the reaction was left to stir for 3 days. The solvent was then removed under reduced pressure. The crude oil that remained was then purified by column chromatography using DCM/MeOH (19:1) as an eluent to give a white solid (31 mg, 7%); ¹H NMR (500 MHz, MeOD) δ 8.74 (1H, s, H-1), 8.58 (1H, s, H-4), 3.19-3.15 (2H, m, H-6), 1.89-1.84 (9H, m, H-8), ¹³C NMR (126 MHz, MeOD) δ 176.56 (C-5), 151.66 (C-4), 151.61 (C-3), 145.74 (C-1), 70.13 (C-6), 25.95 (C-8). HPLC (reverse phase) 0.5 mU/min MeOH/H₂O 90:10 in 12 min, λ=254 nm, Rt=4.72 min (99%). (2R,3S,4R,5R)-2-(hydroxymethyl)-5-(6-(methylamino)-9H-purin-9-yl)tetrahydrofuran-3,4-diol (9a). Methylamine (0.26 mL, 5.86 mmol, 2.8 eq.) and TEA (0.29 mL, 2.08 mmol, 1 eq.) were added to a stirring solution of 6-chloropurine riboside (600 mg, 2.09 mmol, 1 eq.) in EtOH (15 mL). The reaction was then heated to 30° C. for 16 h. The solvent was then removed under reduced pressure. The crude oil that remained was then purified by column chromatography using DCM/MeOH (19:1) as an eluent to give a white solid (274 mg, 73%); ¹H NMR (500 MHz, MeOD) δ 8.23 (2H, s, H-6,9), 5.95 (1H, d, J=6.5 Hz, H-5), 4.74 (1H, dd, J=6.4, 5.1 Hz, H-4), 4.32-4.31 (1H, m, H-3), 4.16 (1H, q, J=2.5 Hz, H-2), 3.90-3.72 (2H, m, H-1), 1.28 (3H, m, H-11); 13C NMR (126 MHz, MeOD) δ 89.89 (C-5), 86.83 (C-2), 74.05 (C-4), 71.32 (C-3), 62.13 (C-1), 7.89 (C-11); LCMS-ES (m/z): Found [M+H]⁺ 282.10, [C₁₁H₁₅N₅O₄H] requires 282.11. HPLC(reverse phase) 0.5 mL/min MeOH/H₂O 90:10 in 12 min, λ=254 nm, Rt=4.73 min (99%).

### (2R,3S,4R,5R)-2-(hydroxymethyl)-5-(6-(isopropylamino)-9H-purin-9-yl)tetrahydrofuran-3,4-diol (9b)

Isopropylamine (0.18 mL, 2.11 mmol, 1 eq.) and TEA (0.29 mL, 2.08 mmol, 1 eq.) were added to a stirring solution of 6-chloropurine riboside (600 mg, 2.09 mmol, 1 eq.) in EtOH (15 mL). The reaction was then heated to 40° C. for 16 h. The solvent was then removed under reduced pressure. The crude oil that remained was then purified by column chromatography using DCM/MeOH (19:1) as an eluent to give a white solid (471 mg, 73%); ¹H NMR (500 MHz, MeOD) δ 8.24 (1H, s, H-6), 8.21 (1H, s, H-9), 5.94 (1H, d, J=6.5 Hz, H-5), 4.74 (1H, dd, J=6.4, 5.1 Hz, H-4), 4.31 (1H, m H-3), 4.16 (1H, m, H-2), 3.81 (2H, m, H-1), 1.31 (6H, d, J=6.5 Hz, H-12); ¹³C NMR (126 MHz, MeOD) δ 152.17 (C-9), 139.98 (C-6), 89.91 (C-5), 86.84 (C-2), 74.05 (C-4), 71.32 (C-3), 62.12 (C-1), 21.43 (C-12): LCMS-ES (m/z): Found [M+H]⁺ 310.13, [C₁₃H₁₉N₅O₄H] requires 310.14. HPLC(reverse phase) 0.5 mL/min MeOH/H₂O 80:20 in 12 min, λ=254 nm, Rt=5.03 min (99%).

### (2R,3R,4S,5R)-2-(6-(benzylamino)-9H-purin-9-yl)-5-(hydroxymethyl)tetrahydrofuran-3,4-diol (9c)

Benzylamine (0.46 mL, 4.19 mmol, 1.5 eq.) and TEA (0.58 mL, 4.19 mmol, 1.5 eq.) were added to a stirring solution of 6-chloropurine riboside (800 mg, 2.79 mmol, 1 eq.) in EtOH (15 mL). The reaction was then refluxed at 80° C. for 16 h. The product crashed out of solution when placed in an ice water bath and stirred vigorously. OL031 was then filtered off and dried under reduced pressure to give a white solid (948 mg, 95%); ¹H NMR (500 MHz, MeOD) δ 8.26 (1H, s, 146), 8.23 (1H, s, H-9), 7.38 (2H, d, J=7.6 Hz, H-13), 7.31 (2H, t, J=7.5 Hz, H-14), 7.24 (1H, t, J=7.3 Hz, H-15), 5.96 (1H, d, J=6.5 Hz, H-5), 4.75 (1H, dd, J=6.4, 5.1 Hz, H-4), 4.32 (1H, dd, J=5.1, 2.5 Hz, H-3), 4.17 (1H, q, J=2.5 Hz, H-2), 3.90-3.73 (2H, m, H-1); ¹³C NMR (126 MHz, MeOD) δ 128.15 (C-14), 127.11 (C-13), 126.84 (C-15), 89.92 (C-5), 86.83 (C-2), 74.05 (C-4), 71.31 (C-3), 62.12 (C-1); LCMS-ES (m/z): Found [M+H]⁺ 358.13, [C₁₇H₁₉N₅O₄H] requires 358.14. HPLC (reverse phase) 0.5 mL/min MeOH/H₂O 80:20 in 12 min, λ=254 nm, Rt=5.34 min (99%).

### (2R,3S,4R,5R)-2-(hydroxymethyl)-5-(6-((4-hydroxyphenethyl)amino)-9H-purin-9-yl)tetrahydrofuran-3,4-diol (9d)

Inosine (500 mg, 1.86 mmol, 1 eq.), tyramine (384 mg, 2.80 mmol, 1.5 eq.) and PyBOP (1.46 g, 2.80 mmol, 1.5 eq.) were dissolved in anhydrous ACN (20 mL) and substoichiometric amounts of DMF (2 ml) under an inert atmosphere. DIPEA (0.65 ml, 3.73 mmol, 2 eq.) was then added slowly over 5 min at FT and the reaction was left to stir for 3 days. The solvent was then removed under reduced pressure. The crude oil that remained was then purified by column chromatography using DCM/MeOH (19:1) as an eluent to give a white solid (347 mg, 48%); ¹H NMR (500 MHz, DMSO) δ 8.34 (1H, s, H-6), 8.24 (1H, s, H-9), 7.04 (2H, d, J=7.3 Hz, H-14), 6.68 (2H, d, J=8.4 Hz, H-15), 5.89 (1H, d, J=6.2 Hz, H-5), 4.63-4.60 (1H, m, H-4), 4.16-4.14 (1H, m, H-3), 3.98-3.96 (1H, m, H-2), 3.70-3.64 (2H, m, H-1) 3.58-3.53 (2H, m, H-11), 2.81-2.78 (2H, m, H-12); ¹³C NMR (126 MHz, DMSO) δ 129.99 (C-14), 115.58 (C-15), 86.37 (C-2), 73.92 (C-4), 71.13 (C-3), 62.14 (C-1), 36.79 (C-12); LCMS-ES (m/z): Found [M+H]⁺ 388.20, [C₁₈H₂₁ NO₅H] requires 388.15. HPLC (reverse phase) 0.5 mL/min MeCN/H₂O 80:20 in 12 min, λ=254 nm, Rt=4.38 min (99%).

### (2R3R4S,5R)-2-(6-(cyclopentylamino)-9H-purin-9-yl)-5-(hydroxymethyl)tetrahydrofuran-3,4-diol (9e)

Inosine (750 mg, 2.80 mmol, 1 eq.), cyclopentylamine (0.41 mL, 4.19 mmol, 1.5 eq.) and PyBOP (2.183 g, 4.19 mmol, 1.5 eq.) were dissolved in anhydrous ACN (20 mL) and substoichiometric amounts of DMF (2 ml) under an inert atmosphere. DIPEA (0.97 ml, 5.59 mmol, 2 eq.) was then added slowly over 5 min at RT and the reaction was left to stir for 3 days. The solvent was then removed under reduced pressure. The crude oil that remained was then purified by column chromatography using DOVI/MeOH (19:1) as an eluent to give a white solid (749 mg, 80%); ¹H NMR (500 MHz, MeOD) δ 8.27 (1H, s, H-6), 8.23 (1H, s, H-9), 5.97 (1H, d, J=6.5 Hz, H-5), 4.76 (1H, dd, J=6.4, 5.1 Hz, H-4), 4.34 (1H, dd, J=5.1, 2.5 Hz, H-3), 4.19 (1H, q, J=2.5 Hz, H-2), 3.92-3.75 (2H, m, H-1), 2.15-2.09 (1H, m, H-11), 1.86-1.61 (8H, s, H-12,13); ¹³C NMR (126 MHz, DMSO) δ 152.84 (C-9), 140.06 (C-6), 88.42 (C-5), 86.37 (C-2), 73.92 (C-4), 71.12 (C-3), 62.14 (C-1), 32.49 (C-11), 23.93 (C-12,13); LCMS-ES (m/z): Found [M+H]⁺ 336.16, [C₁₅H₂₁N₅O₄H] requires 336.16. HPLC (reverse phase) 0.5 mL/min MeOH/H₂O 80:20 in 12 min, λ=254 nm, R1=5.43 min (95%).

### (2R3S,4R,5R)-2-(hydroxymethyl)-5-(6-(neopentylamino)-9K-purin-9-yl)tetrahydrofuran-3,4-diol (9f)

Inosine (300 mg, 1.12 mmol, 1 eq.), neopentylamine (0.20 mL, 1.68 mmol, 1.5 eq.) and PyBOP (873 mg, 1.68 mmol, 1.5 eq.) were dissolved in anhydrous ACN (20 mL) and substoichiometric amounts of DMF (2 ml) under an inert atmosphere. DIPEA(0.39 ml, 2.24 mmol, 2 eq.) was then added slowly over 5 min at RT and the reaction was left to stir for 3 days. The solvent was then removed under reduced pressure. The crude oil that remained was then purified by column chromatography using DCM/MeOH (19:1) as an eluent to give a white solid (305 mg, 81%); ¹H NMR (500 MHz, MeOD) δ 8.27 (1H, s, H-6), 8.20 (1H, s, H-9), 5.95 (1H, d, J=6.5 Hz, H-5), 4.75 (1H, dd, J=6.4, 5.1 Hz, H-4), 4.33 (1H, dd, J=5.1, 2.5 Hz, H-3), 4.17 (1H, q, J=2.5 Hz, H-2), 3.90-3.73 (2H, m, H-1), 3.50-3.46 (2H, m, H-11), 1.00 (9H, s, H-13); ¹³C NMR (126 MHz, MeOD) δ 155.42 (C-10), 152.13 (C-9), 147.66 (C-8), 140.04 (C6), 119.84 (C-7), 89.94 (C-5), 86.86 (C-2), 74.06 (C-4), 71.33 (C-3), 62.13 (C-1), 51.06 (C-11), 31.94 (C-12), 26.28 (C-13); LCMS-ES (m/z): Found [M+H]⁺ 338.18, [C₁₅H₂₃N₅O₄H] requires 338.18. HPLC(reverse phase) 0.5 mL/min MeCN/H₂O 80:20 in 12 min, λ=254 nm, Rt=4.66 min (99%).

### REFERENCES

1. Valente E M, Abou-Sleiman P M, Caputo V, Muqit M M, Harvey K, Gispert S, Ali Z, Del Turco D, Bentivoglio A R, Healy D G, et al: Hereditary early-onset Parkinson's disease caused by mutations in PINKi. Science 2004, 304:1158-1160.
2. Khalil B, E1 Fissi N, Aouane A, Cabirol-Pol M J, Rival T, Lievens J C: PINKi-induced mitophagy promotes neuroprotection in Huntington's disease. Cell Death Dis 2015, 6:e1617.
3. Witte M E, Mahad D J, Lassmann H, van Horssen J: Mitochondrial dysfunction contributes to neurodegeneration in multiple sclerosis. Trends Mol Med 2014, 20:179-187.
4. Reddy P H: Role of mitochondria in neurodegenerative diseases: mitochondria as a therapeutic target in Alzheimer's disease. CNS Spectr 2009, 14:8-13; discussion 16-18.
5. Billia F, Hauck L, Konecny F, Rao V, Shen J, Mak T W: PTEN-inducible kinase 1 (PINK1)/Park6 is indispensable for normal heart function. Proc Natl Acad Sci USA 2011, 108:9572-9577.
6. Liu X, Ye B, Miller S, Yuan H, Zhang H, Tian L, Nie J, Imae R, Arai H, Li Y, et al: Ablation of ALCAT1 mitigates hypertrophic cardiomyopathy through effects on oxidative stress and mitophagy. Mol Cell Biol 2012, 32:4493-4504.
7. Morais V A, Verstreken P, Roethig A, Smet J, Snellinx A, Vanbrabant M, Haddad D, Frezza C, Mandemakers W, Vogt-Weisenhorn D, et al: Parkinson's disease mutations in PINKi result in decreased Complex I activity and deficient synaptic function. EMBO Mol Med 2009, 1:99-111.
8. Wilhelmus M M, van der Pol S M, Jansen Q, Witte M E, van der Valk P, Rozemuller A J, Drukarch B, de Vries H E, Van Horssen J: Association of Parkinson disease-related protein PINKi with Alzheimer disease and multiple sclerosis brain lesions. Free Radic Biol Med 2011, 50:469-476.
9. Fiesel F C, et al.: (Patho-)physiological relevance of PINKI-dependent ubiquitin phosphorylation. EMBO Rep 2015, 16(9):1114-1130.
10. Hou X, et al.: Age- and disease-dependent increase of the mitophagy marker phospho-ubiquitin in normal aging and Lewy body disease. Autophagy 2018, 14(8):1404-1418.
11. Allen, G. F. G., et al.: Loss of iron triggers PINKI/Parkin-independent mitophagy. EMBO Rep. 2013, 14(12):1127-35.
12. McWilliams, T. G., et al.: mito-OC illuminates mitophagy and mitochondrial architecture in vivo. J Cell Biol. 2016, 214(3):333-45.
13. Gan, Z. Y., et al.: Activation mechanism of PINKI. Nature. 2022, 602(7896):328-335.
14. Kakade, P., et al.: Mapping of a N-terminal a-helix domain required for human PINKI stabilization, Serine228 autophosphorylation and activation in cells. Open Biol. 2022, 12(1):210264.

1. A compound of General Formula (I), including all tautomers thereof: 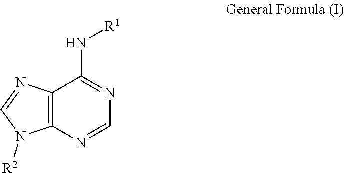
wherein:
R¹ is a C₁₋₁₀ alkyl, C₃₋₁₀ cycloalkyl, C₆₋₁₀ aryl, heterocycloalkyl or heteroaryl group, optionally substituted with one or more substituents selected from OH, halo, nitro, C₁₋₄ alkyl, C₁₋₄ haloalkyl, —O(C₁₋₄ alkyl), —O(C₁₋₄ haloalkyl), NH₂, NH(C₁₋₄ alkyl) and N(C₁₋₄ alkyl)₂; and
R² is a furanose moiety of General Formula (II): 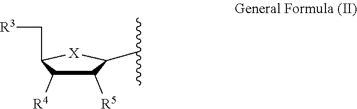
wherein:
X is O, NH, S or CH₂;
R³ is H, OH, halo, nitro, C₁₋₄ alkyl, C₁₋₄ haloalkyl, —O(C₁₋₄ alkyl), —O(C₁₋₄ haloalkyl), NH₂, NH(C₁₋₄ alkyl) and N(C₁₋₄ alkyl)₂, or a mono-, di- or tri-phosphate derivate of General Formula (VIII), wherein q is 0, 1 or 2, and each R¹¹ is independently selected from OH or an aryloxy, amino acid ester or pivaloyloxymethyl masking group; 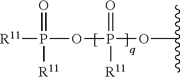
and
R⁴ and R⁵ is independently selected from H, OH, halo, nitro, C₁₋₄ alkyl, C₁₋₄ haloalkyl, —O(C₁₋₄ alkyl), —O(C₁₋₄ haloalkyl), NH₂, NH(C₁₋₄ alkyl) and N(C₁₋₄ alkyl)₂,
or a pharmaceutically or veterinarily acceptable salt or hydrate thereof, for use in the treatment of a neurodegenerative disease or condition that is characterised by an elevated level of Ubiquitin Ser65 phosphorylation and/or cancer.

2. The compound for use according to claim 1, for use in the treatment of idiopathic Parkinson's disease, Lewy body dementia and/or cancer.

3. The compound for use according to claim 1, wherein R1 is a group of General Formula (III-A), (III-B), (III-C), (III-D), (III-E) or (III-F): 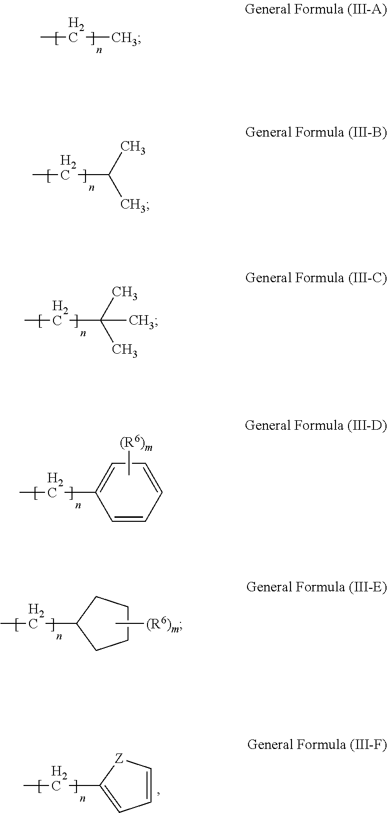
wherein:
n is 0, 1, 2 or 3;
m is 0, 1, 2 or 3;
Z is O, NH, S or CH₂, and is preferably CH₂; and
R⁶ is OH or O(C₁₋₄alkyl).

4. The compound for use according to claim 3, wherein R¹ is selected from:

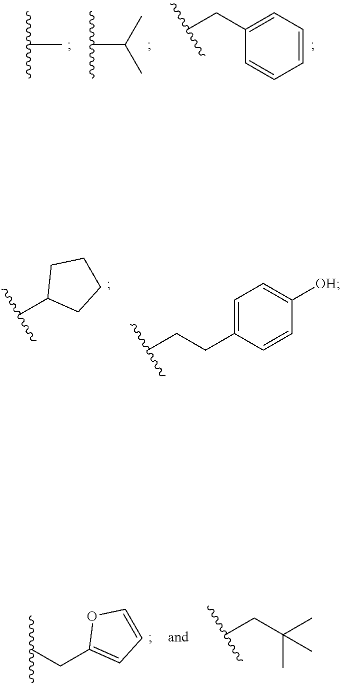

5. The compound for use according to claim 1, wherein X is O, and/or R⁴ and R⁵ are independently selected from H and OH, and/or R³ is H, OH or a mono-, di- or tri-phosphate derivative of General Formula (VIII).

6. The compound for use according to claim 5, wherein each of R³, R⁴ and R⁵ are OH.

7. The compound for use according to claim 5, wherein R³ and R⁴ are both OH, and R⁵ is H.

8. The compound for use according to claim 1, wherein the compound of General Formula (I) is selected from:

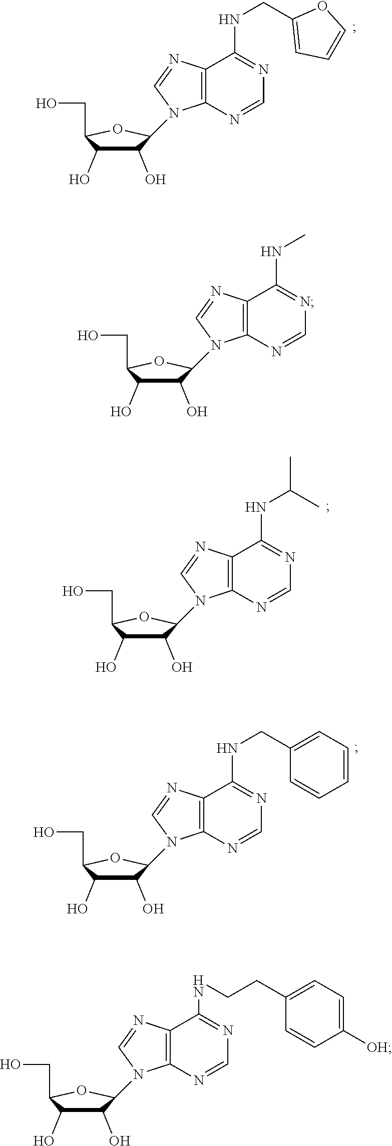

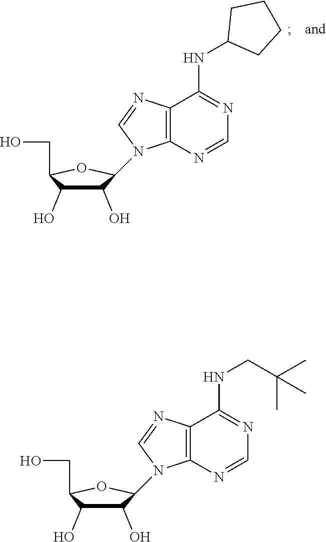

9. A compound of General Formula (I), including all tautomers thereof: 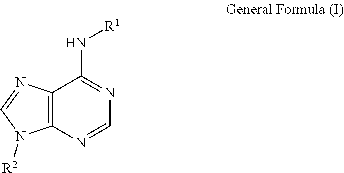
wherein:
R¹ is a C₁₋₁₀ alkyl, C₃₋₁₀ cycloalkyl, C₆₋₁₀ aryl, heterocycloalkyl or heteroaryl group, optionally substituted with one or more substituents selected from OH, halo, nitro, C₁₋₄ alkyl, C₁₋₄ haloalkyl, —O(C₁₋₄ alkyl), —O(C₁₋₄ haloalkyl), NH₂, NH(C₁₋₄ alkyl) and N(C₁₋₄ alkyl)₂; and
R² is a furanose moiety of General Formula (II): 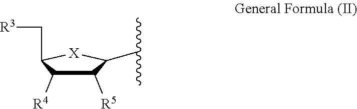
wherein:
X is O, NH, S or CH₂;
R³ is H, OH, halo, nitro, C₁₋₄ alkyl, C₁₋₄ haloalkyl, —O(C₁₋₄ alkyl), —O(C₁₋₄ haloalkyl), NH₂, NH(C₁₋₄ alkyl) and N(C₁₋₄ alkyl)₂, or a mono-, di- or tri-phosphate derivate of General Formula (VIII), wherein q is 0, 1 or 2, and each R¹¹ is independently selected from OH or an aryloxy, amino acid ester or pivaloyloxymethyl masking group; 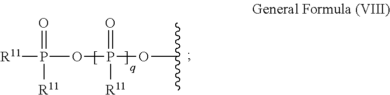
and
R⁴ and R⁵ is independently selected from H, OH, halo, nitro, C₁₋₄ alkyl, C₁₋₄ haloalkyl, —O(C₁₋₄ alkyl), —O(C₁₋₄ haloalkyl), NH₂, NH(C₁₋₄ alkyl) and N(C₁₋₄ alkyl)₂,
or a pharmaceutically or veterinarily acceptable salt or hydrate thereof, provided that the compound is not kinetin riboside.

10. The compound according to claim 9, wherein R¹ is a group of General Formula (III-A), (III-B), (III-C), (III-D), (III-E) or (III-F): 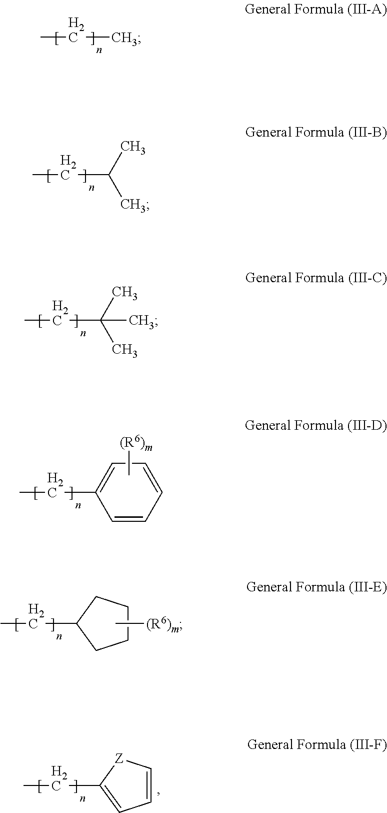
wherein:
n is 0, 1, 2 or 3;
m is 0, 1, 2 or 3;
Z is O, NH, S or CH₂, and is preferably CH₂; and
R⁶ is OH or O(C₁₋₄alkyl).

11. The compound according to claim 9, wherein R¹ is selected from:

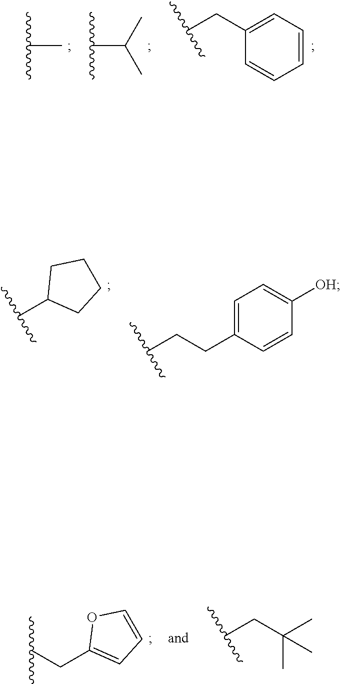

12. The compound according to claim 9, wherein X is O, and/or R⁴ and R⁵ are independently selected from H and OH, and/or R³ is H, OH or a mono-, di- or tri-phosphate derivative of General Formula (VIII).

13. The compound according to claim 12, wherein: each of R³, R⁴ and R⁵ are OH, or wherein R³ and R⁴ are both OH, and R⁵ is H.

14. The compound according to claim 9, selected from:

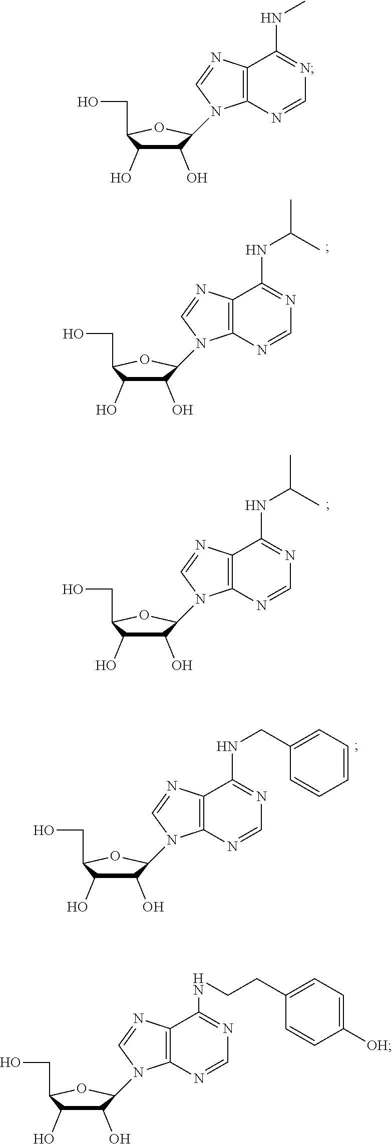

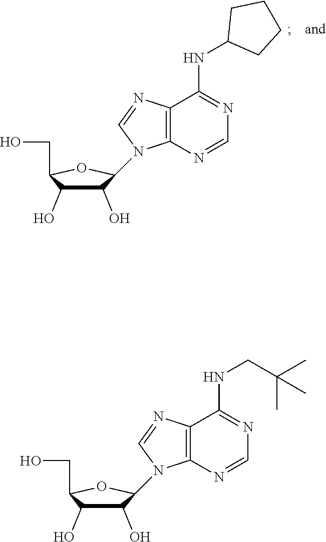

15. A process for the preparation of a compound of General Formula (I) according to claim 9, said process comprising:
(A) reacting a 6-halopurine derivative of General Formula (V) with a nucleophile compound of General Formula (VI): 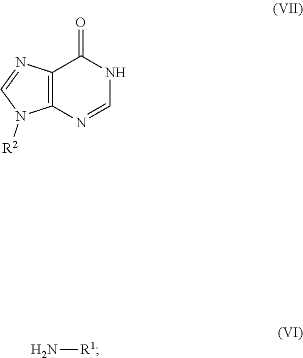
or
(B) reacting a hyphoxanthine derivative of General Formula (VII) with a nucleophile compound of General Formula (VI) in the presence of benzotriazole-1-yloxytripyrrolidinophosphonium hexafluoro phosphate (‘PyBOP’): 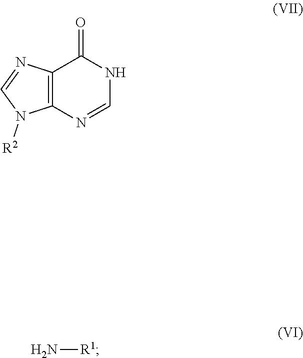
wherein R₇ is halo, preferably chloro, and R₁ and R₂ are as defined for General Formula (I) in any of claims 9 to 14.

16. A compound according to claim 9 or kinetin riboside for use in the preparation of an agent for the treatment of cancer or a disorder or condition that is associated with elevated levels of phosphorylated ubiquitin.

17. A method of treating cancer or a disorder or condition that is associated with elevated levels of phosphorylated ubiquitin, said method comprising administering to a patient in need of such a treatment an effective amount of kinetin riboside or a compound according to claim 9.

18. The method according to claim 17, wherein the disorder or condition that is associated with elevated levels of phosphorylated ubiquitin is selected from idiopathic Parkinson's disease and Lewy body dementia.

19. A pharmaceutical composition comprising kinetin riboside or a compound according to claim 9 and a pharmaceutically or veterinarily acceptable excipient or carrier.

20. The pharmaceutical composition according to claim 19, formulated for oral delivery.

21. A combination therapeutic comprising kinetic riboside or a compound according to claim 9 and an additional therapeutic agent used in the treatment of cancer or a neurodegenerative disease or condition, for simultaneous, separate or sequential use in the treatment of cancer or a neurodegenerative disease or condition that is characterised by an elevated level of Ubiquitin Ser65 phosphorylation.
